(19) United States
(12) Patent Application Publication
(10) Pub. No.: US 2026/0207090 A1
(43) Pub. Date: Jul. 23, 2026

(54) OXYGEN SATURATION MEASUREMENT TECHNOLOGIES

(71) Applicant: Auckland UniServices Limited, Auckland (NZ)

(72) Inventors: Michel NIEUWOUDT, Auckland (NZ); Marco BONESI, Auckland (NZ)

(21) Appl. No.: 19/134,935

(22) PCT Filed: Dec. 5, 2023
(86) PCT No.: PCT/NZ2023/050144

§ 371 Date: Jun. 2, 2025

(30) Foreign Application Priority Data
Dec. 5, 2022 (AU) 2022903699
Aug. 21, 2023 (AU) 2023902642

(51) Int. Cl. A61B 5/1455; A61B 5/00

(52) U.S. Cl. CPC A61B 5/14552; A61B 5/742; A61B 2560/0431; A61B 2562/0238

(57) ABSTRACT

Provided are measurement devices and systems for measuring the blood oxygenation of organs and tissue. Aspects of the technology provide measurement devices and systems which combine multiple forms of spectral analysis, and multiple frequencies of light in order to allow for analysis of blood oxygenation at a range of depths within the organs and tissue.

### 1. STATEMENT OF CORRESPONDING APPLICATIONS

This application claims the benefit of, and priority to Australian Provisional Patent Application No. 2022903699, filed on 5 Dec. 2022, and Australian Provisional Patent Application No. 2023902642, filed on 21 Aug. 2023, both of which are hereby incorporated by reference in their entirety.

### 2. FIELD OF THE TECHNOLOGY

The present technology relates to systems, methods, and devices for measurement of oxygen saturation levels. The present technology may find particular application in measuring the oxygen levels in human and animal blood, tissues and/or organs, however this should not be seen as limiting on the technology.

### 3. BACKGROUND TO THE TECHNOLOGY

Ischaemia is a restriction in the blood supply to a part of the body such as tissues, muscles, or organs. This results in a lack of oxygen supply, which is necessary for tissue to stay alive and heal. Ischaemia is a common consequence of surgical procedures on the body. For example, surgical anastomoses (joints) may breakdown and leak with an inadequate blood supply. There is therefore a need to be able to monitor the blood oxygen saturation in tissues, muscles and organs as Ischaemia has a significant health and economic burden.

By way of example, pancreatic cancer is a common solid cancer with a rising incidence and poor survival statistics. Resection of a cancer in the head of the pancreas may require a complex operation (Whipple's procedure) that entails four anastomoses. The pancreatic neck is known to be a watershed region, meaning that it falls between two sources of blood and is prone to ischaemia. A common complication of the operation is a leak from the pancreatic anastomosis, leading to postoperative pancreatic fistula (POPF) resulting in spillage of digestive enzymes, abscess, haemorrhage and, in a proportion of patients, death. Ischaemia often contributes to the failure of the pancreatic anastomosis, which occurs in 30-40% of patients. Despite decades of research the incidence of POPF has remained largely unchanged and a stubborn and unacceptable rate of POPF remains. The detection of ischaemia is currently made on the basis of visual inspection of the cut pancreatic surface prior to the formation of the anastomosis, which itself induces some ischaemia with the placement of sutures. However, ischaemia is an important risk factor in almost all surgical specialties.

Currently a typical way to assess tissue vascularity/extent of ischaemia in tissue during surgery is by sight such as checking for discolouration of tissue. For plastic surgical skin flap procedures, surgeons are able to inject a patient with a fluorescent dye and then use a fluorescence camera to monitor the blood flow. However, this is time consuming and can result in up to 20-minute delays. Furthermore, the technology is cumbersome, bulky, and not commonly used during surgeries.

Blood-oxygen monitoring is currently performed using pulse oximetry to detect oxygen saturation (SpO₂) levels; a technology which is now present in some modern smart watches and fitness trackers. Pulse oximetry systems generally work by shining a light source onto the tissue of the person or animal, and analysing the reflected light, or light passing through the tissues. Oxyhaemoglobin (O₂Hb) and deoxyhaemoglobin (HHb) absorb red, and near-infrared (IR) light differently, and the amounts of light absorbed fluctuates during the user's cardiac cycle, allowing the pulse oximetry sensors to detect only the arterial blood.

Tissue oxygen saturation measurements (StO₂) can be performed on veins and capillaries, using a technique known has Near Infra-Red Spectroscopy (NIRS).

Existing pulse oximetry devices, however, can generally only measure the blood-oxygen levels near the surface of the tissue/skin/organ by having a light source located on a first side of the tissue/organ and a light receiver on the opposing side of the tissue/organ. In some applications, such as during surgery, it may be impractical to include separate source and receiver as it can be desirable for the blood oxygen sensor to be as compact as possible in order to enable highly localized tissue oxygen saturation measurement and easy to reposition to analyse different areas of the tissue/organ.

Some existing systems for measuring tissue oxygen saturation use probes, for example in the form of pads, that are too large to be able to be used in many surgical procedures, such as for example many gastro-intestinal, pancreatic and brain surgeries.

### 4. OBJECT OF THE TECHNOLOGY

It is an object of the technology to address any one or more of the foregoing issues.

Alternatively, it is an object of the technology to provide blood-oxygen measurement technologies which allow for deep tissue/organ blood oxygen measurements.

Alternatively, it is an object of the technology to provide blood-oxygen measurement technologies which allow for surface blood oxygen measurements together with deep tissue measurements.

Alternatively, it is an object of the technology to provide a compact blood-oxygen measurement probe.

Alternatively, it is an object of the technology to provide a blood-oxygen measurement probe for highly localized tissue oxygenation measurements.

Alternatively, it is an object of the technology to at least provide the public with a useful choice.

### 5. SUMMARY OF THE TECHNOLOGY

According to one aspect of the technology there are provided systems, methods and/or devices for the measurement of blood oxygen saturation levels.

According to another aspect of the technology, there are provided systems, methods and/or devices for measuring blood oxygen saturation levels in human and/or animal tissues and/or organs.

According to another aspect of the technology, there are provided systems, methods and/or devices for measuring blood oxygen saturation levels in human or animal tissues at a depth of between 1 mm and 30 mm.

According to another aspect of the technology, there is provided a transmission medium for use in a measurement device, the transmission medium comprising a lens configured to focus received light into one or more signal acquisition units.

According to another aspect of the technology, there is provided a measurement system, comprising a measurement device configured for use in measuring oxygen saturation levels using both Raman spectroscopy and visible and/or near infrared spectroscopy.

According to another aspect of the technology, there is provided a measurement device for use in a measurement system, the measurement device comprising:
a housing,
a set of openings with transmission media at a first end of the housing; and
a plurality of light guides within the housing, the plurality of light guides configured to connect to at least one stimulus generator, and at least one signal acquisition unit,
   wherein the measurement device is configured to receive light from the stimulus generator through one or more of the plurality of light guides, and pass the light through the transmission medium to expose an organ and/or tissue to the light, and wherein the measurement device is further configured to receive light from the organ and/or tissue and pass the received light through one or more of the light guides to transmit the received light to the at least one signal acquisition unit.

According to another aspect of the technology, there is provided a measurement device for use in measuring oxygen saturation levels in an organ and/or tissue, the measurement device comprising:
a housing having a first end and a second end, the first end being distal to the second end, and the housing comprising:
   a first opening at the first end, the first opening comprising one or more transmission media,
   a first set of light guides configured to receive a first source of light, and direct the first source of light through the one or more transmission media and out of the first opening,
   a second opening at the first end, the second opening comprising one or more transmission media,
   a second set of light guides configured to receive a second source of light, and direct the second source of light through the one or more transmission media of the second opening and out of the second opening,
   a third set of light guides configured to receive a third source of light through the second opening, and to direct the third source of light into a signal acquisition unit and/or processor configured to perform a Raman spectral analysis of the third source of light,
   a third set of one or more openings at the first end, the third set of one or more openings comprising one or more transmission media,
a fourth set of light guides configured to receive a fourth source of light through the third set of one or more openings, and to direct the fourth source of light, into a signal acquisition unit and/or processor configured to perform visible and/or near-infrared spectral analysis of the fourth source of light.

Throughout the present specification, unless the context clearly requires otherwise, reference to a “set” should be understood to include a set containing any number of articles, including for example a set of one, or a set of more than one articles.

In examples, the first source of light may comprise one or more wavelengths between 700 nm and 900 nm.

In examples, the first source of light may comprise one or more wavelengths of: between approximately 700 nm and approximately 800 nm, between approximately 795 nm and approximately 825 nm, and/or between approximately 815 nm and approximately 900 nm.

In examples, the first source of light may have wavelengths of approximately 785 nm, approximately 810 nm and/or approximately 830 nm.

In examples, the first set of light guides may comprise a plurality of light guides, for example a first light guide of the plurality of light guides may be configured to receive light having a wavelength of between approximately 700 nm and approximately 800 nm, a second light guide of the plurality of light guides may be configured to receive light having a wavelength of between approximately 795 nm and approximately 825 nm, and a third light guide of the plurality of light guides may be configured to receive light having a wavelength of between approximately 815 nm and approximately 900 nm.

In examples, the fourth source of light used in the visible and/or near infrared spectral analysis may substantially arise through an interaction of the organ and/or tissue with the first source of light.

In examples, the second source of light may have a wavelength of between approximately 380 nm and approximately 425 nm.

In examples, the second source of light may have a wavelength of approximately 410 nm.

In examples, the third source of light used in the Raman spectral analysis may substantially arise through an interaction of the organ and/or tissue with the second source of light.

In examples, one or more of the transmission media may comprise a lens, such as a plano-convex lens. In examples, one or more of the transmission media may comprise a window.

In examples, one or more of the transmission media may comprise a filter.

In examples, the first, second, third and/or fourth light guides may comprise one or more optical fibres. In examples, the first set of light guides may comprise two or more optical fibres.

In examples, the first set of light guides may comprise three optical fibres.

In examples, the third set of light guides may comprise three or more optical fibres.

In examples, the third set of light guides may comprise six optical fibres.

In examples, the fourth set of light guides may comprise three or more optical fibres.

In examples, the fourth set of light guides may comprise five optical fibres.

In examples, the first source of light and the second source of light may be provided by one or more stimulus generators.

In examples, the stimulus generators may each have a power of less than approximately 50 mW.

In examples, the stimulus generators may each have a power of substantially 30 mW.

In examples, the one or more stimulus generators may be laser(s).

In examples, the one or more stimulus generators are laser diode(s).

In examples, the Raman spectral analysis and/or the visible and/or near infrared spectral analysis may be used to determine the oxygen saturation levels in the tissue or organ.

In examples, the measurement device may further comprise a display configured to provide an indication of the oxygen saturation levels.

In examples, the measurement device may be a handheld device.

In examples, the measurement device may be connected to physically and/or remotely to a data collection and/or processing unit.

In examples, the first end may be configured to be positioned in contact with, or in close proximity to, the organ and/or tissue in use, wherein an area of the first end may be 100 mm² or less.

In examples, the first end may have a length and a width, and wherein the length of the measurement tip is 20 mm or less.

In examples, the first end may have a length of between 20 mm and 35 mm.

In examples, the width of the first end may be 5 mm or less.

In examples, the width of the first end may be between 5 mm and 25 mm.

In examples, the third set of light guides may be configured to receive the third source of light through the second opening, and to direct the third source of light, towards and out of the second end of the measurement device into the signal acquisition unit and/or processor configured to perform Raman spectral analysis of the third source of light.

In examples, the fourth set of light guides may be configured to receive the fourth source of light through the third set of one or more openings, and to direct the fourth source of light, towards and out of the second end of the measurement device into the signal acquisition unit and/or processor configured to perform visible and/or near-infrared spectral analysis of the fourth source of light. In examples, one or more of the transmission media comprises a lens. For example, the lens may be a plano-convex lens.

In the examples, one or more of the transmission media comprises a window. For example, the window may be a flat window made of sapphire or quartz. For example, the transmission window may be between approximately 0.5 mm and approximately 3.5 mm long, such as approximately 3 mm long.

In the examples, one or more of the transmission media comprises a filter. For example, the filter might filter out spectral portions of the light that is directed to the signal acquisition unit and/or processor.

In examples, the lens may be configured to direct the received light from the organ and/or tissue into one or more light guides or otherwise direct the light towards the signal acquisition unit.

In examples, the lens may be, configured to direct the received light corresponding to the second source of light into the third set of light guides.

In the examples, the light associated with any one source might pass through more than one transmission window and/or more than one type of transmission window. For example, the light corresponding to one source might pass through one lens and one filter.

In examples, the light guides may comprise one or more optical fibres.

In examples, the second set of light guides and the third set of light guides may comprise a first optical sub-assembly. In examples, the first set of light guides and the fourth set of light guides may comprise a second optical sub-assembly.

In examples, the housing may be substantially elongate, or otherwise have a length which is at least 2 times greater than its width. For example, the housing may have a width of 20 mm or more preferably less than 10 mm.

In examples, the housing may have a substantially cylindrical shape, for example the housing may be a substantially hollow narrow cylinder.

In examples the housing may be constructed of a metal. For example the housing may be constructed of a steel, such as a surgical or medical-grade stainless steel such as Austenitic 304 or 316 stainless steels.

In examples, the housing may comprise any one or more of the stimulus generator(s), signal acquisition unit(s), processor(s), or display.

In examples the measurement device may further comprise any one or more of:
A button configured to turn the measurement device on or off, or otherwise trigger the taking and/or recording of an oxygen saturation measurement.
A power source configured to power the device, such as a battery.
A communications interface configured to transfer one or more of light, power, or data between the measurement device and a measurement system.

In examples, the measurement device may comprise an end cap at a second end of the housing. For example, the communications interface may be configured to pass through the end cap.

In examples, the first source of light and the second source of light may be provided by one or more stimulus generators.

In examples, the stimulus generator(s) may be configured to generate light of at least one wavelength. In some examples the stimulus generator may be configured to generate light of a plurality of wavelengths.

In examples, the stimulus generator(s) may be configured to generate light having a wavelength of one or more of:
Between approximately 380 nm and approximately 425 nm such as approximately 410 nm;
Between approximately 700 nm and approximately 800 nm such as approximately 785 nm;
Between approximately 795 nm and approximately 825 nm such as approximately 810 nm; and
Between approximately 815 nm and approximately 900 nm such as approximately 830 nm.

In examples, the stimulus generators may be configured to generate light using at least one diode, such as a laser diode. For example, the laser diode may be configured to have a power of approximately 50 mW or less, such as substantially 30 mW.

In examples where the stimulus generator(s) is/are configured to generate a plurality of wavelengths, the stimulus generator may be configured to generate and or transmit the plurality of wavelengths sequentially, i.e., one after the other.

In examples where the stimulus generator(s) is/are configured to generate a plurality of wavelengths, the stimulus generator may be configured to generate and or transmit the plurality of wavelengths at spatially distinct locations at the exit port of the holder, i.e. in a fibre bundle.

In examples, the signal acquisition unit may comprise any one or photodiodes, entrance slits, lenses, mirrors, transmission or reflecting gratings (such as a diffraction grating), filters (such as holographic notch filters) charge coupled device (CCD) detectors, and linear arrays.

In examples the display may comprise an LCD, television, monitor, segmented display, coloured lights, interactive GUI or LEDs.

In examples the processor may comprise an application specific integrated circuit (ASIC), microprocessor, or computer processor.

In examples, the processor may be configured to perform one or more of Raman spectral analysis (spectroscopy) and/or infrared and/or near infrared spectral analysis (spectroscopy) on the received light information to determine organ and/or tissue oxygen saturation.

In examples, the measurement system may be configured to perform Raman spectroscopy to determine the oxygen saturation levels at and near the surface of the organ and/or tissue.

In examples, the measurement system may be configured to perform infrared and/or near infrared spectroscopy to determine the oxygen saturation levels at a depth below the surface of the organ and/or tissue. For example, infrared and/or near infrared spectroscopy may be used to determine the oxygen saturation levels at a depth of between approximately 1 mm and 20 mm below the surface of the organ and/or tissue.

In examples the measurement devices/systems may comprise one light guide configured to transfer a wavelength of light to the organ and/or tissue, and more than one light guide configured to receive light from the organ and/or tissue.

In examples, the measurement device may comprise a measurement tip having a tip end configured to be positioned in contact with, or in close proximity to, the organ and/or tissue in use, wherein an area of the tip end is 100 mm² or less. The tip end may have a length and a width, and the length of the measurement tip may be 20 mm or less. The width of the tip end may be 5 mm or less.

According to another aspect of the technology, there is provided a measurement system for measuring oxygen saturation levels in an organ and/or tissue, the system comprising:
a measurement device which comprises:
   a housing; and
   one or more ports with one or more transmission media located at a first (distal) end of the housing,
one or more stimulus generators;
a signal acquisition unit;
a processor; and
a display,
   wherein the stimulus generators are configured to generate light, and transmit the light through the housing and the transmission media, to expose the tissue and/or organ to the different wavelengths of light in use, and wherein the light received back from the tissue and/or organ is configured to pass through the transmission media, and be received by the signal acquisition unit to provide information about the received light, and wherein the information about the received light is passed to at least one processor for determining the oxygen saturation levels in the tissue and/or organ, and wherein the tissue oxygen saturation levels are displayed on the display.

In examples, the measurement device may be a measurement device according to any one or more other aspects of the technology.

According to another aspect of the technology, there is provided a method of measuring blood oxygen levels in an organ and/or tissue, the method comprising the steps of:
A) generating one or more light sources from a stimulus generator;
B) transmitting the light sources to the organ and/or tissue;
C) receiving light from the organ and/or tissue, and passing the received light to a signal acquisition unit to obtain information about the received light;
D) processing the received information using at least one processor to determine one or more blood oxygen levels within the organ and/or tissue.

According to another aspect of the technology, there is provided a measurement device for use in measuring oxygen saturation levels in an organ and/or tissue, the measurement device comprising:
a housing having a first end and a second end, the first end being distal to the second end, and the housing comprising:
a first set of light guides configured to receive a first source of light, and direct the first source of light through one or more transmission media and out of the first end,
a second set of light guides configured to receive a second source of light, and direct the second source of light through one or more transmission media and out of the first end,
a third set of light guides configured to receive a third source of light, and to direct the third source of light into a signal acquisition unit and/or processor configured to perform a Raman spectral analysis of the third source of light,
a fourth set of light guides configured to receive a fourth source of light, and to direct the fourth source of light into a signal acquisition unit and/or processor configured to perform visible and/or near-infrared spectral analysis of the fourth source of light.

In examples, the housing may comprise a first opening at the first end, and the one or more transmission media through which the first source of light is directed may be positioned in the first opening.

In examples, the housing may comprise a second opening at the first end, wherein the one or more transmission media through which the second source of light is directed may be positioned in the second opening.

In examples, the housing may comprise a third opening at the first end, wherein one or more transmission media through which the third source of light is directed may be positioned in the third opening.

In examples, the housing may comprise a fourth opening at the first end, wherein one or more transmission media through which the fourth source of light is directed may be positioned in the first opening.

In examples, the measurement device may further comprise a plurality of fourth openings.

In examples the transmission media through which the first, second, third or fourth sources of light are directed may be the same transmission media through which another of the first, second, third or fourth sources of light are directed.

Further aspects of the technology, which should be considered in all its novel aspects, will become apparent to those skilled in the art upon reading of the following description which provides at least one example of a practical application of the technology.

### 6. BRIEF DESCRIPTION OF THE DRAWINGS

One or more embodiments of the technology will be described below by way of example only, and without intending to be limiting, with reference to the following drawings, in which:

FIG. 1 Shows one example of a measurement device in use on an organ according to the present technology;

FIG. 2 Shows a block diagram of a measurement device in accordance with one example of the present technology;

FIG. 3A Shows a block diagram of a measurement system comprising a measurement device in accordance with one example of the present technology;

FIG. 3B Shows a block diagram of a measurement system of FIG. 3A with the communications interface shown connecting to the transmission media;

FIG. 4 Shows a further example of a measurement device according to another example of the present technology;

FIG. 5A Shows an example of how light can be transferred from a stimulus generator and a transmission medium, and from a transmission medium to a signal acquisition unit according to one example of the technology;

FIG. 5B Shows an alternative example how light can be transferred from a stimulus generator and a transmission medium, and from a transmission medium to a signal acquisition unit according to another example of the technology;

FIG. 6 Shows a timing diagram for sequencing various stimulus sources according to one aspect of the technology;

FIG. 7 Shows a comparison of the wavelength versus transmission percent for a range of transmission media in accordance with the present technology;

FIG. 8 Shows a side view of a transmission media and lens within a measurement device according to one example of the technology;

FIG. 9 Shows one example of a measurement system in use according to one example of the present technology;

FIG. 10 Shows one example of the resolved bands for oxyhaemoglobin, deoxyhaemoglobin, and methaemoglobin, using Raman spectral analysis according to one example of the technology;

FIG. 11 Shows one example layout of stimulus generators and signal acquisition units, or light guides therefore within a measurement device for Raman spectroscopy according to one example of the present technology;

FIG. 12A Shows an example of the light scattering effect in an organ and/or tissue using near infrared light frequencies according to one example of the technology;

FIG. 12B Shows one example layout of stimulus generators and signal acquisition units, or light guides therefore within a measurement device for near infrared spectroscopy according to one example of the present technology;

FIG. 13 Shows one example layout of stimulus generators and signal acquisition units, or light guides therefore within a measurement device for Raman and near infrared spectroscopy according to one example of the present technology;

FIG. 14 Shows an alternative example layout of stimulus generators and signal acquisition units, or light guides therefore within a measurement device for Raman and near infrared spectroscopy according to one example of the present technology;

FIG. 15A shows a perspective view of a measurement device in accordance with another example of the present technology;

FIG. 15B shows an exploded perspective view of the first (distal) end of the measurement device of FIG. 15A;

FIG. 16 Shows an example transmission media for a measurement system comprising a measurement device configured for Raman and near-infrared spectroscopy;

FIG. 17 Shows an alternative example transmission media for a measurement system comprising a measurement device configured for Raman and near-infrared spectroscopy;

FIG. 18A shows an example of a measurement device in accordance with one example of the technology; and

FIG. 18B shows another example of a measurement device in accordance with another example of the technology.

### 7. BRIEF DESCRIPTION OF EXEMPLARY FORMS OF THE TECHNOLOGY

### 7.1. Overview of the Technology

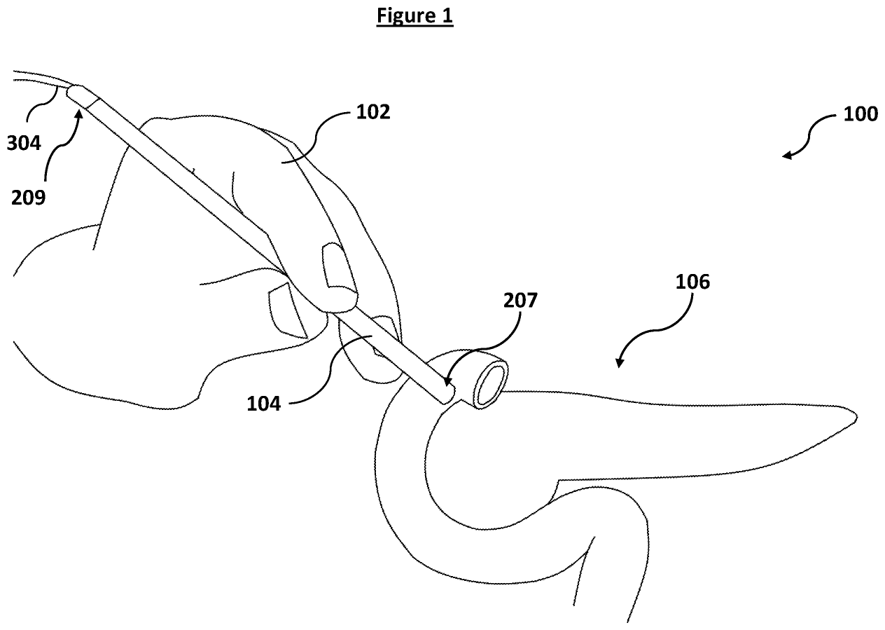

The present technology relates to blood oxygen measurement technologies and devices. FIG. 1 shows one example of the present technology, wherein a user 102 is directing a measurement device 104, onto engagement with an organ and/or tissue 106 in order to determine blood oxygen levels within the organ tissue 106.

Throughout the present specification reference is made herein to organs and tissues, this should be understood to include any part of a body of a human or animal that performs a specific function. Including but not limited to skin, tissue, muscles, liver, heart, kidneys, pancreas etc.

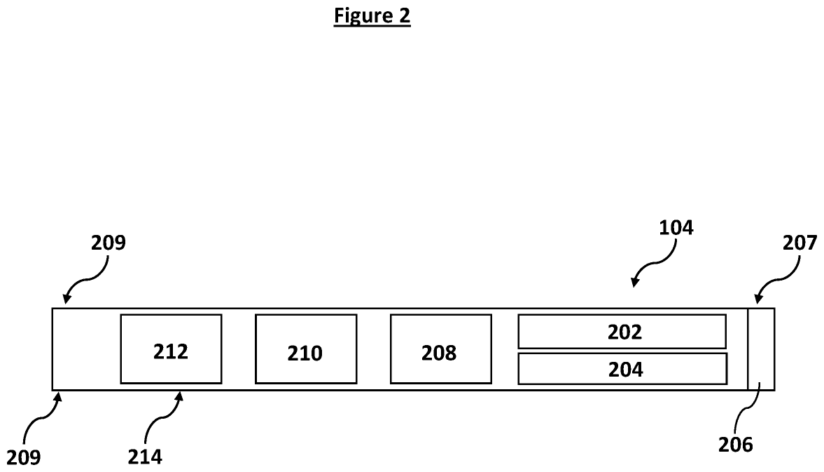

FIG. 2 shows a block diagram overview of one embodiment of a measurement device 104 according to the present technology. In general terms the measurement device 104 comprises:
A stimulus generator 202;
A signal acquisition unit 204;
At least one transmission medium 206;
A processor 208;
A display 210; and
A power source 212.

Each of these components are discussed in greater detail below, but each will first be described in general terms.

The stimulus generator 202 may be configured to generate a stimulus, for example the stimulus generator 202 may be a light source which generates light that is transmitted through at least one transmission medium 206 onto and/or into the organs/tissues 106 of a patient in use.

The signal acquisition unit 204 may be configured to receive signals from the patient, such as reflected and refracted/scattered stimulus signals received back through the transmission medium 206. These signals may then be communicated to the processor 208 for processing. In some examples of the technology, the signal acquisition unit 204 and processor 208 may be provided as a single module or component and are shown in FIG. 2 as separate components for sake of explanation only.

The processor 208 may be configured to process the signals provided from the signal acquisition unit in order to determine the likely oxygen levels (SpO₂ and/or StO₂) within the organ/tissue 106. Once the likely oxygen levels have been determined, these may be presented to the user via the display 210.

In some examples of the technology, the measurement device 104 may include an internal power source 212 such as a battery, which allows the components described herein to be powered in use.

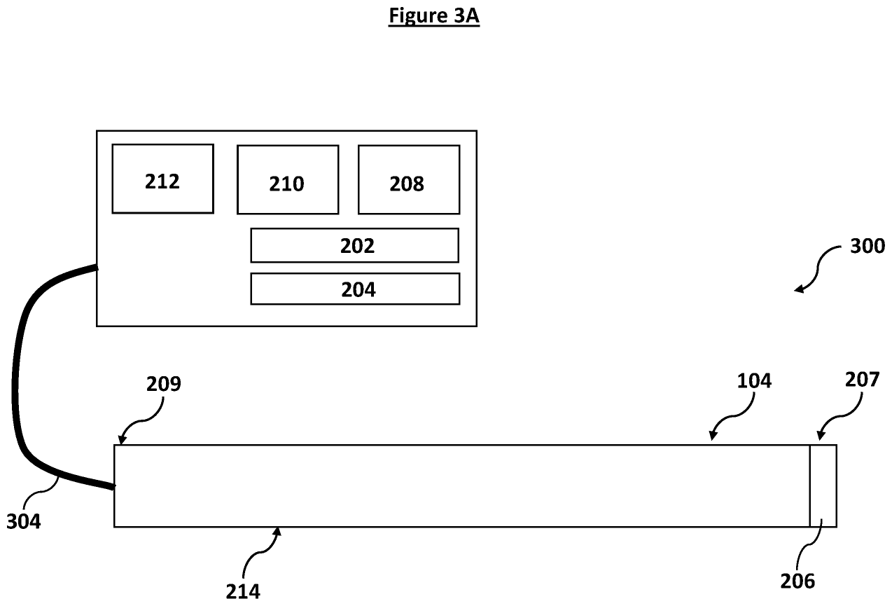

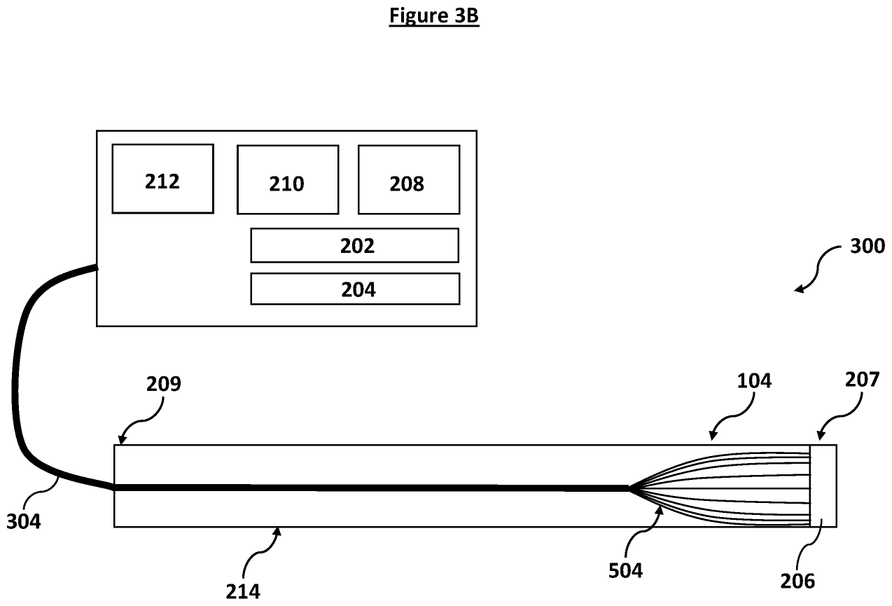

In the example of FIG. 2, the measurement device 104 is configured to be a portable, stand-alone unit. In other words, the device may be used to determine blood oxygen levels without needing to be connected to any external equipment. This however should not be seen as limiting on the technology, and in an alternative example such as the one shown in FIG. 3, any one or more of the stimulus generators 202, signal acquisition unit 204, processor 208, display 210 and/or power source 212, may be external to the measurement device.

For example, in FIG. 3, the measurement device 202 is part of a measurement system 300, in which the stimulus generator 202, signal acquisition unit 204, processor 208, display 210 and power source 212 are external to the measurement device 104. For example, the measurement device 104, may be operatively connected to the stimulus generator 202, and signal acquisition unit 204, via a communications interface 304. In the example of FIGS. 3A and 3B the communications interface is primarily configured to direct light between the stimulus generator(s) 202 and transmission medium 206 at the first end of the housing 214, and direct light which is received through the first end 207 of the housing into the signal acquisition unit 204 and processor 208. Accordingly, in some examples the communications interface may be configured to communicate light and may comprises a plurality of light guides in the form of fibre optic cables.

In the examples of FIG. 3A, the communications interface 304, is shown as connecting to the second end 209 of the measurement device 104, substantially opposite to the first (distal) end 207 comprising the transmission medium/media 206. However, it should be appreciated that where the communications interface 304 is configured to transfer light through the measurement device, the communications interface 304 may extend through the body of the measurement device, such that the light guides 504 contained therein can be coupled to the transmission media at the first end 207 of the measurement device 104 as shown in FIG. 3B.

It should be appreciated that while the examples shown illustrate the communications interface 304 passing through the second end 209 of the measurement device 104, in alternative examples this may instead be configured to pass through a side wall of the housing.

In the example of FIGS. 3A and 3B, the measurement device 104 comprises a transmission medium 206, which in use transfers the stimulus from the stimulus generator to the organ/tissue 106, receives the stimulus from the organ/tissue, and transfers the received stimulus to the signal acquisition unit 204. For example, where optical stimulus is used, the stimulus may be transferred between the measurement device and other components of the measurement system using one or more optical fibres, i.e., the communications interface 304 also comprises one or more optical fibres. Alternatively, the stimulus may be processed or measured within the measurement device using one or more processors 208, and the processed measurement communicated over the communications interface 304 using any methods known to those skilled in the art, including wired and wireless communications.

In some examples of the technology, the communications interface further comprises two or more conductors configured to transfer power to or from the measurement device.

### 7.1.1. Measurement Device Construction

As shown in FIGS. 1 to 3, the measurement device 104 may comprise an elongate housing 214 with the transmission medium 206 located at a first end 207 of the housing 214. This first end 207 may otherwise be known as a measurement end or distal end of the housing 214, in that it is distal from a second end 209 of the housing.

It may be advantageous for the measurement device 104 to have an elongate profile in order to reach organs/tissues within a human or animal patient, while minimising the size of any incisions needing to be made in the patient, and while allowing localized measurement of tissue.

The first (distal) end 207 may provide a measurement tip configured to be positioned in contact with, or in close proximity to, the organ and/or tissue in use. The size of the first (distal) end 207 may be configured according to the intended use of the measurement device 104. The first (distal) end 207 may have a maximum area that faces outward from the measurement device 104, i.e. a tip end area that, in use, contacts or is in close proximity to the organ/tissue being measured. In certain forms, the area of the tip end may be sufficiently small that the tip is able to be positioned against the appropriate organ/tissue. In some scenarios, for example some surgical procedures, it may be beneficial for the measurement tip to be sufficiently small to be inserted into the body and against the appropriate tissue. In addition, the tissue may have a small area. It is advantageous to be able to measure even small areas of organ/tissue so a surgeon can conserve as much healthy organ/tissue as possible. In some forms, the area of the tip end may be no more than approximately 100 mm², or less than this area in some forms, for example approximately 80 mm².

The measurement device 104 may otherwise be known as a “probe” or “measurement probe”, as used in this specification, the terms may be used interchangeably. For sake of consistency, the present specification refers to the “probe” as a measurement device 104 herein.

In examples of the technology, it may be advantageous for the measurement device 104 to be a handheld device, usable using a single hand of a user. In some applications such as in surgical applications, one handed use may allow for the user to perform other actions while taking measurements, such as moving tissue, or other organs/tissues 106 aside to get better access to the organ being tested. For example, the measurement device 104 may have a construction which allows the user to accurately probe the tissues/organs 106 of interest, such as the elongate constructions described herein.

In one example of the technology, the measurement device may have a substantially cylindrical construction having a diameter of less than 20 mm or more preferably less than 10 mm such as less than 5 mm. Use of a cylindrical construction should not be seen as limiting on the technology, and in some examples described herein the transmission material may have a rectangular or trapezoidal shape. In other examples, the measurement device may have any suitable cross-sectional shape including circular, oval, square, or any polygonal shape. For example, the measurement device 104 may be substantially rectangular, and the first end 207 of the measurement device may have a height of between approximately 1 cm and approximately 2 cm, such as approximately 1.6 cm, and a width of between approximately 0.5 cm and approximately 1.5 cm, such as approximately 1 cm.

In one example the measurement device may have a length in a longitudinal direction of between approximately 10 cm and approximately 30 cm, such as approximately 15 cm to 25 cm or approximately 20 cm.

The housing 214 of the measurement device may comprise a material which is easy to sterilise and/or disinfect. For example, the housing 214 of the measurement device may be constructed from a metal such as a steel, for example a surgical/medical-grade stainless steel such as Austenitic 304 or 316 stainless steels. In other examples, the housing 214 may comprise carbon steel, aluminium, titanium, a polymer, or elastomer.

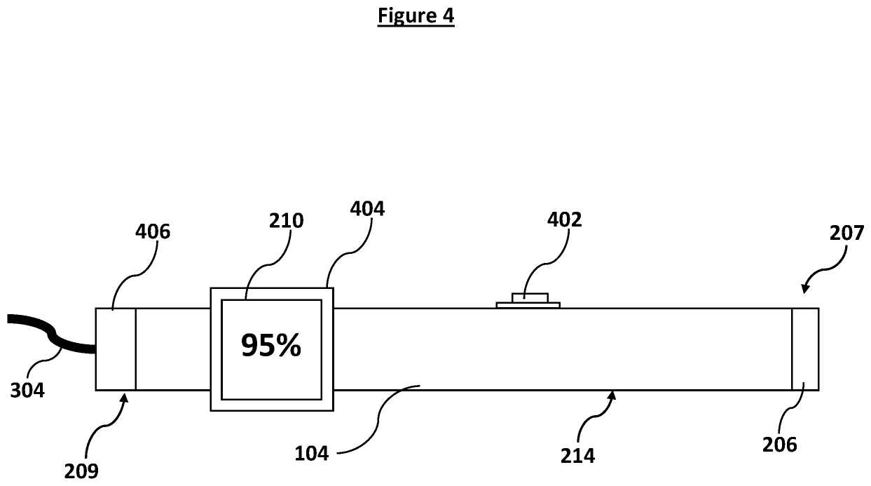

In some examples of the technology, such as those shown in FIG. 4, the measurement device 4 may further comprise one or more inputs 402 such as a buttons or switches configured to turn the measurement device on/off or to trigger the taking and/or recording of tissue oxygenation measurements.

In some forms, the housing 214 may have a width which varies along its length. For example, the housing may increase in diameter towards the second end 209 of the housing relative to the first (distal) end 207 of the housing. In the example of FIG. 4 for example the measurement device 104 comprises a display housing 404, comprising a display 210. The display housing 404 may be part of the measurement device housing 214 or attached to the measurement device housing. Alternatively, or additionally, the width of the housing 214 may increase towards the second end, for example to allow for more room within the measurement device for internal components such as those described in relation to FIG. 2.

The housing 214 of the measurement device 104 may further comprise an end cap 406 proximate to the second end 209 of the measurement device 104 as shown in FIG. 4. The end cap may, for example, have a threaded engagement with the housing 214, such as an external thread which engages with an internal thread in the housing 214. In other examples the end cap may comprise clips, a press-fit connection, or be attached to the housing 214 using adhesives.

In some examples the housing may comprise a plurality of materials. For example, in FIG. 4, the end cap 406 may comprise a polymer or elastomer while the rest of the housing may comprise a metal such as steel. Use of a polymer or elastomer in the end cap 406 may advantageously allow for a hermetic seal between the housing 214, and the communications interface 304.

### 7.1.2. Stimulus Generator

In one example of the technology, the stimulus generator 202 comprises one or more light generators 502 configured to generate light. In one example the light is generated within the spectrum of visible/near infrared light, such as between 750 nm and 850 nm. In one example light may be generated with a wavelength of between approximately 380 nm and approximately 425 nm, in other words a light in the violet/blue portion of the visible light spectrum.

More preferably the light generator(s) 502 may be configured to generate a light with a wavelength of approximately 410 nm. Use of light with a wavelength of approximately 410 nm may be particularly beneficial in measuring oxygen saturation using Raman spectroscopy which should be familiar to those skilled in the art but will be described in greater detail herein.

In another example of the technology, the stimulus generator 202, comprises one or more light generators 502 configured to generate light with a wavelength of between approximately 750 nm and approximately 850 nm, in other words a light in the near infrared light spectrum.

More preferably the light generator(s) 502 may be configured to generate a light with a wavelength of approximately 785 nm. Use of light with a wavelength of approximately 785 nm may be particularly beneficial in measuring oxygen saturation using near infrared spectroscopy which should be familiar to those skilled in the art but will be described in greater detail herein.

More preferably the light generator(s) 502 may be configured to generate a light with a wavelength of approximately 810 nm. Use of light with a wavelength of approximately 810 nm may be particularly beneficial in measuring oxygen saturation using near infrared spectroscopy which should be familiar to those skilled in the art but will be described in greater detail herein.

More preferably the light generator(s) 502 may be configured to generate a light with a wavelength of approximately 830 nm. Use of light with a wavelength of approximately 830 nm may be particularly beneficial in measuring oxygen saturation using Raman spectroscopy which should be familiar to those skilled in the art but will be described in greater detail herein.

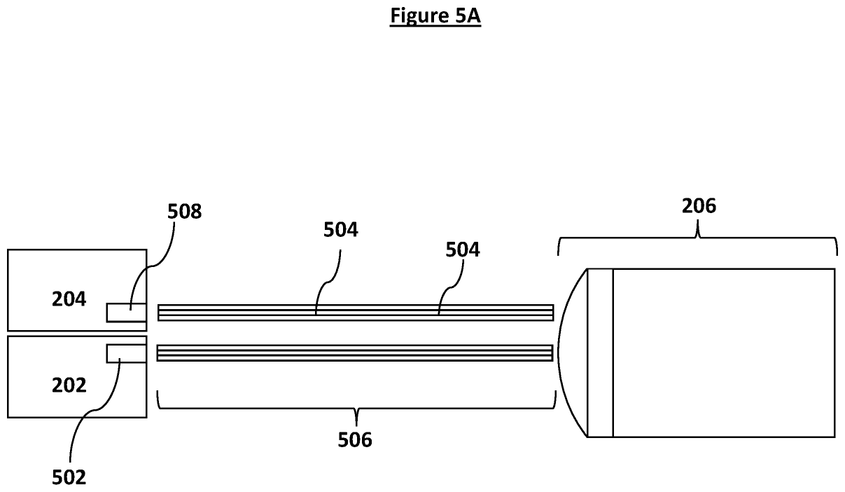

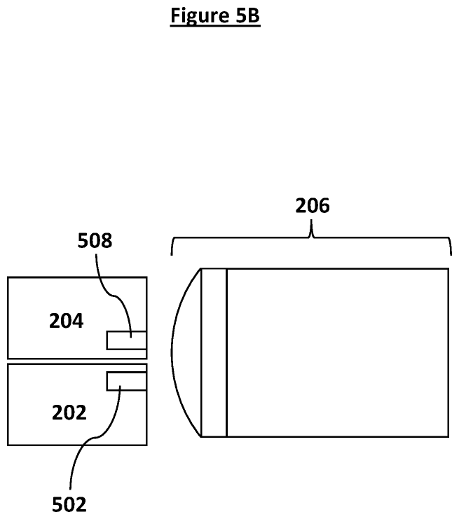

In examples, such as FIGS. 5A and 5B, and the stimulus generator 202 may comprise one or more light generator(s) 502 in the form of lasers such as laser diodes. In one example shown in FIG. 5A, the light generated by the light generator(s) is transferred to the transmission medium 206 using one or more light guides 504 or optical fibres. For example, one or more light guides or fibres may be used to transfer the light generated by each of the one or more light generator(s) 502.

In some examples of the technology, the stimulus generator may be configured to generate a plurality of stimulus signals, such as a plurality of different frequency/wavelength light sources. In these examples the stimulus generator may be configured to generate the stimulus sources simultaneously, while in other examples, the stimulus generator may be configured to generate each of the stimulus sources sequentially, such that only a single stimulus is present at any one time.

In some examples, the light guides 504 may also be configured to transmit light received from the organ/tissue 106 and transfer the light to a signal acquisition unit 204, as described herein. For example, the light may be detected by one or more detection components 508, such as photodiodes, gratings (such as a diffraction grating), charge coupled device (CCD) detectors, and linear arrays.

In some examples of the technology, for each stimulus generator and associated signal acquisition unit and/or processor, a first set of light guides 504 may be provided for transferring the light from the stimulus generator to the organ or tissue, and a second set of light guides may be provided for transferring the light received from the organ or tissue, back to the associated signal acquisition unit and/or processor.

In examples of the technology comprising two or more stimulus generators, a first set of light guides may be provided to transfer the light from the first stimulus generator to the organ/tissue, and a second set of light guides may transfer the received light from the organ/tissue to the signal acquisition unit and/or processor. A third set of light guides may be provided to transfer the light from the second stimulus generator to the organ/tissue, and a fourth set of light guides may transfer the received light from the organ/tissue to the signal acquisition unit and/or processor.

Each set of light guides may comprise any number of individual light guides, for example between one light guide and 50 light guides. For example, each set of light guides may comprise any number of optical fibres configured for use as light guides.

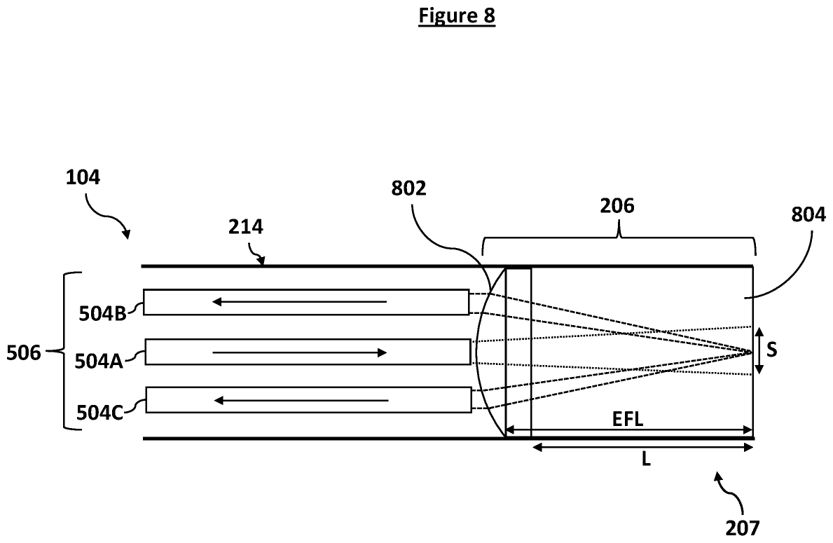

In some examples of the technology, the measurement device 104 may comprise one or more optical sub-assemblies 506. The optical subassemblies 506 may comprise light guides 504 configured to transmit light from a stimulus generator onto organs and/or tissue and light guides configured to receive light from the organs and/or tissue and transfer the received light to signal acquisition units/processors described herein. For example, the first optical subassembly 506 may be configured, dimensioned, or arranged for use in performing one or more types of spectral analysis, such as Raman spectroscopy or near infrared spectroscopy. The first (distal) end 207 of the fibre that contacts the tissue may also comprise one or more filters configured to remove unwanted wavelengths of light. In some examples, the first (distal) end 207 can also include one or more transmission windows 804, for example it may be advantageous for one of the optical subassemblies 506, such as the Raman fibre sub-assembly, to include one or more of a filter and a transmission window 804, as shown in FIG. 8. In some examples, it may be advantageous to use filters to ensure that only the returned light from the light source is detected by the signal acquisition units. For example, when the light source used has a wavelength close to that of visible light, it may be beneficial to filter out most of the visible light spectrum, so that the signal acquisition unit can focus on the frequencies corresponding to the light source. For example, in some applications of the technology a light source having a wavelength of approximately 410 nm may be used. As 410 nm light overlaps with the visible light spectrum (380 nm to 700 nm) it may be advantageous to use a filter, such as a bandpass filter to attenuate light wavelengths received which are either side of the desired 410 nm band.

In some examples of the technology, the measurement device 104 may comprise a first optical sub-assembly configured for use with Raman spectroscopy, and a second optical sub-assembly configured for use with near infrared spectroscopy.

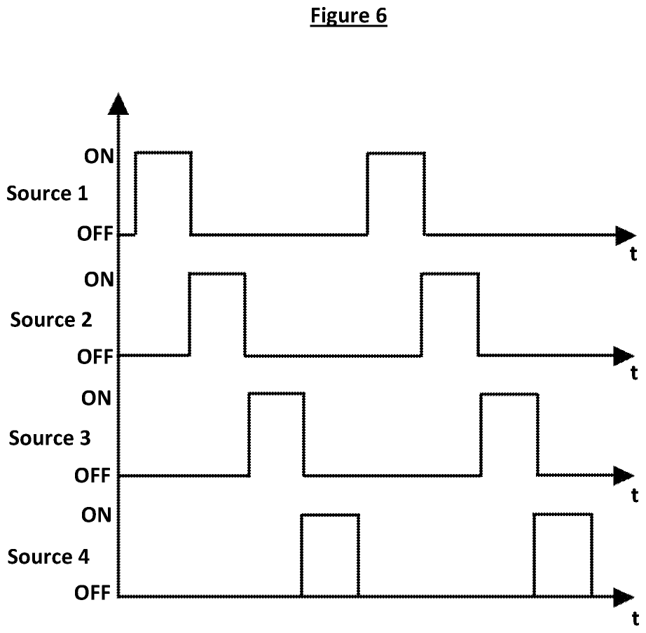

FIG. 6 shows one example of a stimulus generator configured to generate four different stimuli in the form of different frequencies of light. In the illustrated example, four light generators 502 are used labelled source 1 to source 4. These light sources are activated sequentially such that only one light source is active at a time, which can help to prevent cross contamination of the signals received at the signal acquisition unit described herein. In the illustrated example:
Source 1 is a light source with a wavelength of approximately 785 nm;
Source 2 is a light source with a wavelength of approximately 830 nm;
Source 3 is a light source with a wavelength of approximately 850 nm; and
Source 4 is a light source with a wavelength of approximately 410 nm;

However, this combination or sequencing of light sources should not be seen as limiting on the technology, and any combination of light sources may be used, with any suitable combinations of relative sequencing or timing. For example, where a combination of signal acquisition units is used, the timing and duration of the corresponding light source activation may be varied in order to ensure each of the signal acquisition units has sufficient time to process and determine a measurement with a desired level of accuracy.

For example, sources 1, 2 and 3 may correspond to a signal acquisition unit 204 tasked with performing a near infrared spectral analysis, while source 4 may correspond to a signal acquisition unit 204 tasked with performing Raman spectrum analysis, and accordingly it may be desirable for the ‘on’ duration of source 4 to be greater than the ‘on’ duration for sources 1 to 3.

### 7.1.3. Transmission Medium

The transmission medium or media 206 described herein may provide an interface between the stimulus (light) generated by the stimulus generator and the organs/tissues 106 of the patient in use. In addition, the interface may allow for incident light to be detected by the signal acquisition methods described herein.

The transmission medium/media 206 may be constructed of one or more components, for example a single transmission medium 206 may be provided which is common to one or more of the light guides. Alternatively, a plurality of transmission media 206 may be provided, including for example at least one transmission media per light guide, or at least one transmission media which is common to two or more light guides. For example, in some forms, a single transmission media may be provided, which is common to the near infrared light sources, and each of the light guides which receive the reflected/refracted infrared light may have a common transmission media or one or more transmission media per light guide. Similarly, the transmission media 206 associated with Raman spectroscopy may comprise transmission media common to the light guides optically connected to the Raman light source, as well as the light guides configured to receive the reflected/refracted Raman light for analysis.

The transmission medium/media described herein may be constructed from any suitable light transmission material. It may be desirable for the transmission medium to comprise a material with a low nonlinear refractive index, and a transmission range wide enough to cover the range of frequencies which are desirable to be transmitted and received by the measurement device 104.

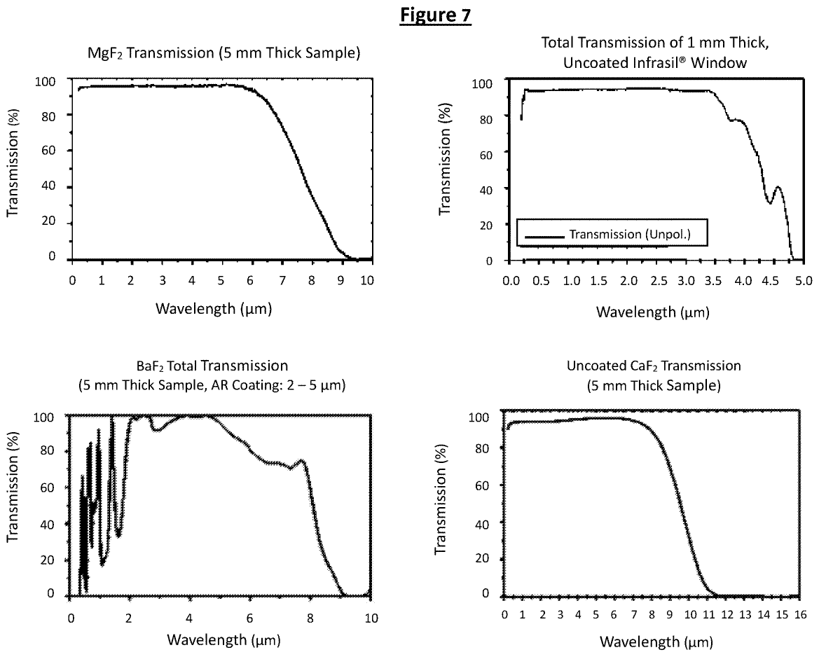

FIG. 7 shows examples of the transmissibility of various transmission media over a range of wavelengths of light. It should be appreciated that Infrasil® is a brand name for an optical quartz material. In particular it can be seen that for wavelengths between 0.41 μm (410 nm) and 0.85 μm (850 nm), magnesium fluoride may be advantageous for use with the present technology.

In some examples of the technology, the transmission medium 206 provides separation between the internal components of the measurement device 104 and the organs/tissues 106 of the patient. For example, it can be advantageous for the transmission medium 206 to provide a hermetic seal between the housing and transmission medium in order to prevent or limit the ingress of contaminants and/or bodily fluid into the measurement device 104.

In certain forms, the transmission medium/media 206 may be housed in a housing, which may itself be opaque and include openings therethrough within which the transmission medium/media 206 is/are retained.

Some forms of the technology described in this specification may include one transmission medium while other forms may include more than one transmission media. In places, these alternative forms are described collectively and, to help with readability, one of the terms “medium” or “media” may be used out of convenience, instead of using both terms. It should be understood that the selection of the term “medium” or “media” is therefore not necessarily an indication that the form of the technology being described has one transmission medium or multiple transmission media (as the case may be). Instead, it should be understood that, unless the context clearly requires there to be a single transmission medium or multiple transmission media, the described form of the technology may comprise either one or many transmission media.

### 7.1.3.1. Transmission Lenses

In some examples of the technology, it can be advantageous for the transmission medium 206 to comprise a lens 802 to aid in focusing or defocusing the outgoing light from the measurement device 104, and incoming light received by the measurement device 104. While in other examples the transmission medium may be substantially planar, or otherwise substantially perpendicular to the longitudinal axis of the measurement device so as to provide minimal deflection of the outgoing or incoming light. In yet further examples described herein it may be advantageous to use a transmission medium which comprises a substantially planar region and a lensed region. For example, this may be advantageous when combining multiple tissue oxygenation detection technologies within a single measurement device 104.

FIG. 8 shows one example of the technology in which the measurement device 104 comprises a transmission medium 206 comprising a lens 802, and a transmission window 804. As illustrated the measurement device 104 comprises a plurality of light guides which couple the outgoing light from the stimulus generator(s) to the transmission medium 204, and the incoming light from the transmission medium to the signal acquisition unit(s).

The lens may be constructed of any suitable material known to those skilled in the art, including for example sapphire and diamond. Sapphire and diamond in particular can be beneficial in the present technology due to being one of the hardest optical materials and being substantially transparent to the wavelengths of light being used in the present technology.

In the illustrated example, a central light guide 504A is configured to transmit a stimulus to the transmission medium 206 from a stimulus generator 202 (not shown in FIG. 8), and the outer light guides 504B are configured to receive light from the transmission medium 206 and transfer the light to the signal acquisition unit 204 (not shown in FIG. 8). It should be appreciated that while FIG. 8 shows an internal arrangement of a measurement device 104 in a two-dimensional perspective, any number of light guides may be used, for example the central outgoing light guide may be surrounded by any number of incident light guides such as three or more, or more preferably five or more.

In the illustrated examples, the lens is a convex lens, such as a plano-convex lens. Use of a plano-convex lens may advantageously aid in converting light incident from a spot on the organ/tissue 106 to parallel light rays which can be received by the signal acquisition unit, for example through optical fibres, or other light guides.

It may be advantageous in some forms for the stimulus provided by the stimulus generator to be substantially aligned perpendicular to the lens along the principal axis of the lens, such that the lens provides minimal deflection of the outgoing stimulus.

Similarly, it may be advantageous for the effective focal length (EFL) of the lens to substantially match the length of the transmission medium, such that the spot ‘S’ i.e., illuminated area created by outgoing light onto the organ/tissue 106 is located substantially at the same point on the organs/tissues 106 of the patient. This can allow the light detected from the surface of the organ/tissue to be received by the signal acquisition unit at a maximum intensity, by creating parallel pathways for the light into the light guides 504 or receivers within the signal acquisition unit(s).

In some forms, the length of the lens may be in the region of approximately 1 to 2 mm. In other forms, the length of the lens may be between approximately 50 μm and approximately 250 μm wide, such as approximately 100 μm.

### 7.1.3.2. Transmission Windows

Another feature of certain forms of the present technology is the use of transmission windows 804 in order to allow for dispersion of the outgoing light from the stimulus generator, to create a larger spot ‘S’ on the surface of the organ/tissue 106. Accordingly, the length of the window ‘L’ can be adjusted to adjust the spot size, and/or allow for lenses with a different focal length.

For example, the transmission windows 804 may be constructed from magnesium fluoride (MgF2), barium fluoride (BaF2), calcium fluoride (CaF2) or quartz.

By way of example, the inventors have determined that, for a spot size ‘S’ of approximately 1 mm, an approximately 2 mm long transmission window 804 may be preferred, while for a spot size ‘S’ of approximately 1.2 mm diameter, an approximately 3 mm long transmission window may be preferred. In other forms, the length of the transmission window 804 may be up to approximately 5 mm. Due to the lens having a relatively thin construction, the divergence of the outgoing light is largely due to refraction in the transmission window material which in this example is a magnesium fluoride crystal. In this way, using a longer transmission window 804 allows the outgoing stimulus (light) to expand/diverge more than is possible with conventional measurement devices. For example, in conventional laser spot measurement devices, the projected spot size can be approximately 0.6 mm in size.

It may be advantageous to provide a relatively large spot size in some examples of the technology, in order to allow for an increased stimulus generator power. For example, a 30 mW stimulus generator (such as a laser) may be used in order to obtain a good signal to noise ratio, while keeping the power density at the surface of the organ/tissue 106 to stay within the maximum permitted exposure (MPE) limits for skin.

In forms in which the length ‘L’ of the transmission window is greater, it can be desirable for the radius of curvature of the lens to be selected to also increase the effective focal length (EFL) as should be familiar to those skilled in the art.

### 7.1.4. Signal Acquisition

The present technology may comprise one or more signal acquisition units, configured to convert received stimulus (light) from the transmission medium into electronic readings which can be processed to determine information about the received stimulus. Suitable signal acquisition units and techniques should be familiar to those skilled in the art, and therefore we do not discuss these in detail here for sake of brevity, but these should be understood to include detection components such as gratings (such as a diffraction grating), charge coupled device (CCD) detectors, and linear arrays.

### 7.1.5. Processor

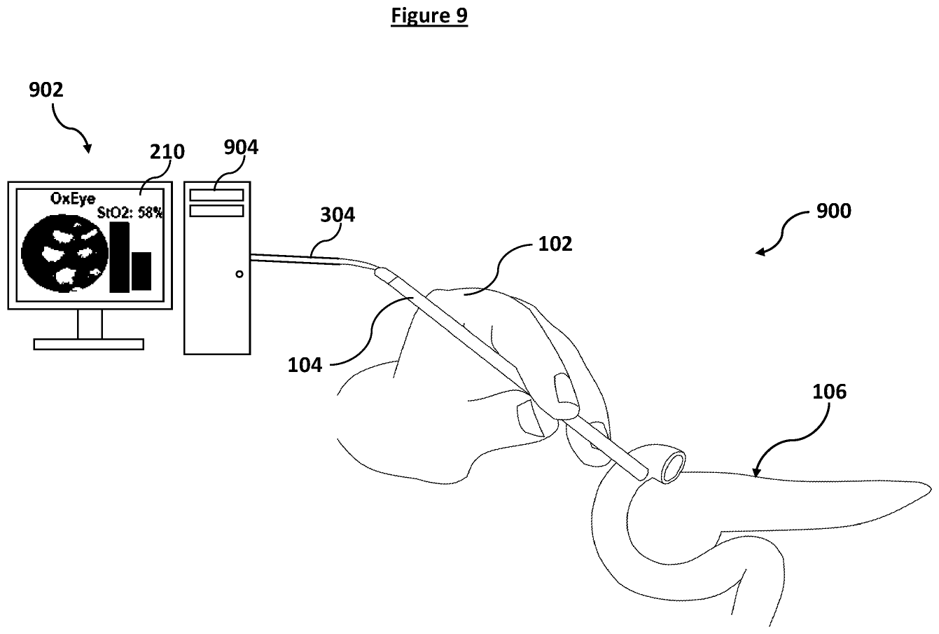

In examples of the technology such as the example shown in FIG. 9, the measurement system or device 104 may comprise at least one processor 902 configured to perform spectral analysis on the signals received by the signal acquisition unit 204. For example, the processor may be configured to perform any one or more of: Raman spectroscopy; near infrared spectroscopy, ultraviolet and visible spectroscopy.

In some examples of the technology, the processor may be an application specific integrated circuit (ASIC), microprocessor, computer processor, or any other suitable processor known to those skilled in the art.

In some examples the processor may be configured to present the spectral analysis on the display 210. In some examples the processor may be configured to record or store the spectral analysis information on computer readable storage 904, such as a hard drive, solid state drive or removable storage device.

### 7.1.6. Raman Spectroscopy

Raman spectroscopy should be known to those skilled in the art. However, in context of the present technology, Raman spectroscopy may be used to perform any one or more of:
Curve resolution of the deoxy and oxy-haemoglobin bands at approximately 1357 and 1375 cm⁻¹ as well as the methaemoglobin band at approximately 1366 cm⁻¹, respectively, and calculate the StO₂ from the ratio. For example, the ratio may be recalibrated prior to testing; and
Partial Less Squares (PLS) regression of the Raman spectra between 550 and 1700 cm⁻¹ for blood oxygen measurement.

In preferred examples of the technology, resonance Raman spectroscopy is used for accurate blood oxygen measurement down to depths of between 0.1 mm and 1 mm.

The use of Raman spectroscopy systems, as described herein, may allow the use of higher laser powers (with lower power density at the tissue) thereby enabling high Raman signals to be received and processed in a very short time.

In examples of the technology in which Raman spectroscopy is used, the measurement device 104 may include lens 802 and transmission window 804 preferably provided within the housing 214 of the measurement device 104. Other features common to Raman spectroscopy systems, such as bandpass filters, may also be used as should be familiar to those skilled in the art. For example, a filter may be provided to filter the wavelengths of light transmitted to the organ or tissue 106, for example a filter may be used between the stimulus generator and the transmission medium, between the transmission medium and the organ or tissue 106 and between the organ or tissue 106 and the detection systems 502 and 508.

In one example of the technology a measurement device 104 may be provided for performing Raman spectra analysis. In this example the measurement device 104 may be configured to provide a spot size ‘S’ of between approximately 1.5 mm and approximately 0.8 mm such as 1.2 mm, on the surface of the organ/tissue 106. For example, this may be implemented using a transmission window 804 of between approximately 2 mm and approximately 3.5 mm, such as approximately 3 mm, and a lens with a diameter of between approximately 2 mm and approximately 3.5 mm, such as approximately 3 mm. In this example it may be advantageous for the lens to have an effective focal length (EFL) such that the focal point of the lens is approximately equal to an end of the transmission window 804.

With a spot size of approximately 1.2 mm, a stimulus generator with a laser supplying powers of between 20 and 50 mW may be used in order to obtain a good signal to noise ratio, while keeping the power density at the surface of the organ/tissue 106 to stay within the maximum permitted exposure (MPE) limits for skin.

For example, with a 410 nm stimulus or light source, the MPE is 1.1 C_{A}t^{0.25} J·cm⁻² where C_{A}=1.1. For an exposure time of 300 milliseconds the energy exposure is 0.8141 J·cm⁻². With a spot size ‘S’ of 1.2 mm the area irradiated on the organ/tissue is 0.011304 cm2, which equates to a limit of 30.67 mW for the laser power.

Accordingly, in one example of the technology it may be preferred to use a stimulus generator 202 configured to generate light sources, wherein the stimulus generator 202 comprises an approximately 30 mW light generator 502 (when measured at the surface of the organ/tissue), and is configured to generate light with a wavelength of any one or more of 410 nm, 785 nm, 810 nm and 830 nm, each with activation/integration times of approximately 300 ms.

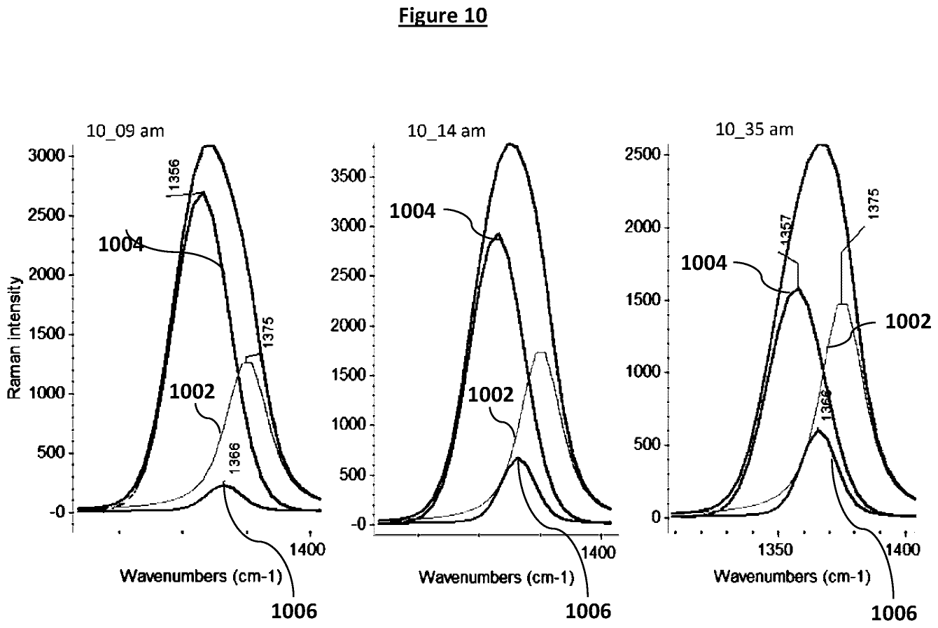

The inventors have found that with these power and activation times, the resulting spectra have a high signal to noise ratio adequate for accurate measurement of the oxy-and deoxy-haemoglobin ring stretching vibrational mode between 1300-1400 cm⁻¹ as shown in FIG. 10. The illustrated graphs, include the resolved bands for oxyhaemoglobin 1002, deoxyhaemoglobin 1004, and methemoglobinemia 1006. In one example, a laser excitation at a wavelength of 410 nm has been used to resonantly enhance the stretching vibration of the porphyrin rings in the Raman spectrum of haemoglobin that occur between 1357-1380 cm⁻¹. This has been found to result in a strongly enhanced Raman peak at 1358 cm⁻¹ (for de-oxygenated haemoglobin, or HHb) and 1375 cm⁻¹ (for oxygenated haemoglobin, or HbO2).

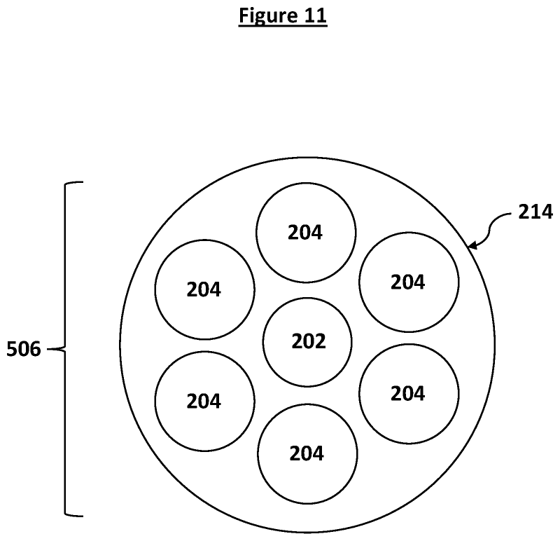

FIG. 11 shows one example arrangement of the stimulus generator, and signal acquisition pathways configured for use with Raman Spectroscopy. In other words, the optical sub-assembly 506 may be associated with Raman spectroscopy. In this example a central stimulus generator 202, such as light generator 502, (or light guide (504) optically connected to a light generator) provides a stimulus source, such as the 410 nm wavelength light described here to an organ/tissue 106 (not shown). Surrounding the central stimulus generator 202, are a plurality of signal acquisition units 204, or light guides 504 optically connected to one or more signal acquisition units 204. This arrangement of stimulus generator 202, signal acquisition units 204 and/or light guides 504, can be used in combination with any one or more of the transmission media 206 described herein, including the lenses 802 and transmission windows 804 thereof and bandpass filters. For example, the lens 802 and transmission window 804 described in relation to FIG. 8.

### 7.1.7. Near Infrared Spectroscopy

Near infrared spectroscopy techniques should be known to those skilled in the art. However, in context of the present technology, near infrared spectroscopy may be used to perform any one or more of:
Analysis of absorption ratios at 785 nm, 810 nm, and 830 nm from near infrared signals in deeper tissue; and
PLS regression of the absorption spectra from NIR/Visible signals in deeper tissue.

The use of near infrared spectroscopy may in some examples enable measurement of the oxygen saturation levels at a greater depth within the tissue or organ 106 of the patient than the Raman or pulse oximetry techniques described herein, while allowing for the stimulus and signal acquisition unit to be optically connected to the same side of the organ/tissue 106.

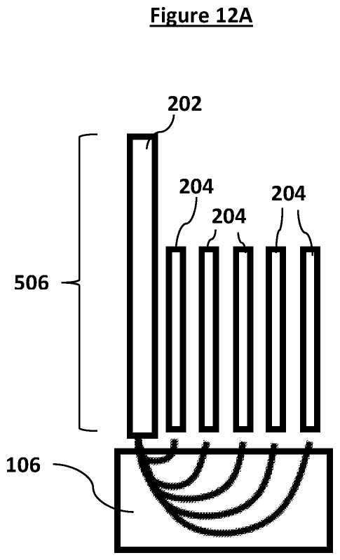

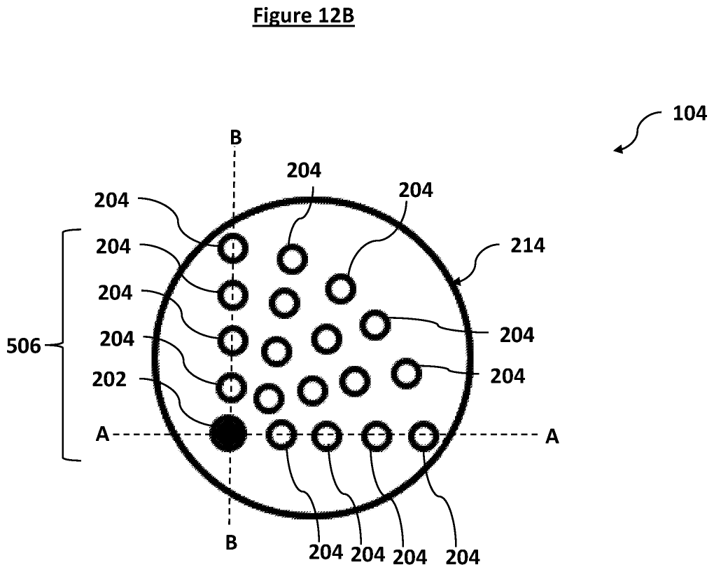

FIG. 12A shows one example of how near infrared spectroscopy may be used to determine the oxygen saturation levels at a depth within the tissue/organ 106. In this example a stimulus generator 202 is provided which provides a light stimulus to the tissue, such as the 785 nm, 810 nm and 830 nm wavelength light sources described herein.

In this example, a plurality of signal acquisition units 204 are provided, or light guides 504 optically connected to one or more signal acquisition units. The signal acquisition units 204 are positioned at increasing distances from the stimulus generator 202, to thereby provide an optical sub-assembly 506 as described herein. Wherein the stimulus generators positioned furthest from the stimulus generator 202, are able to better measure the light scattering in the tissue/organ 106 at greater depths.

It is believed that the depth of light scattered depends on its wavelength, and the signal acquisition unit(s) optically positioned furthest from the stimulus generator 202 influences the depth at which the measurements are taken. For example, if the signal acquisition unit(s) 204 are optically positioned 20-50 mm from the stimulus generator 202, it may be possible to measure down to a depth of approximately 20-30 mm in the tissue/organs 106. Measurement to this depth may be sufficient for assessing tissue viability. However, it can be advantageous to minimise the total size of the measurement device 104, particularly in surgical applications. Accordingly, in some aspects of the technology, it can be advantageous to provide a probe with a first end 207 (tip) having a length (which may be the maximum dimension) measuring no more than 20 mm or more preferably approximately 16 mm.

One configuration of how the stimulus generator(s) 202 and signal acquisition unit(s) 204 may be configured for use with near infrared spectroscopy is shown in FIG. 12B. In this example the stimulus generator(s) 202, is positioned to one side of the measurement device, and the signal acquisition unit(s) 204 are provided with increasing radial separation from the stimulus generator 202. This arrangement may advantageously allow for measurements at a plurality of depths within the tissue, and the relative positioning of the signal acquisition unit(s) 204 to the stimulus generator(s) 202 may advantageously allow measurements to be acquired in a plurality of axes.

For example, in the arrangement shown, a series of signal acquisition unit(s) 204 are provided in a first axis ‘A’, while a second series of signal acquisition unit(s) 204 are provided in a second axis ‘B’ the first axis being different to the second axis, such as approximately perpendicular to the first axis, or approximately at 90 degrees to the first axis. Furthermore, in the arrangement shown, one or more additional signal acquisition unit(s) 204 are arranged between the first and second axes, so as to provide a cone of coverage on the organ/tissue 106. It should be appreciated FIG. 12B represents one example arrangement for a substantially circular probe configuration, and that not all signal acquisition unit(s) 204 have been labelled for sake of clarity, but like symbols represent like features. In other examples of the technology, such as where polygonal measurement devices are used, the arrangement of signal acquisition unit(s) 204 and stimulus generators 202 may be varied accordingly, but preferably in a way which substantially increases separation distance between at least one of the signal acquisition unit(s) 204 and the stimulus generator 202 to provide an increased tissue/organ 106 measurement depth.

Use of a plurality of light guides and/or signal acquisition units, may also be useful in increasing the total amount of light, which is received by the measurement devices described herein, and therefore may be beneficial in taking accurate measurements of the oxygen levels within the tissues.

In measurement devices comprising near infrared spectroscopy, the use of lenses 802 may not be required, and instead a substantially planar transmission medium may be used. Furthermore, the depth of the transmission window may be reduced so as to more closely couple the signal acquisition unit(s) 204 and stimulus generators 202 to the surface of the organ/tissue 106. In other words, for near infrared spectroscopy, the transmission window may simply be configured to act as a hermetic seal between the housing 214 of the measurement device 104, so as to prevent or reduce the ingress of fluids or contaminants into the measurement device 104.

In some examples of the technology, the Raman spectral analysis and near infrared spectral analysis described here may be combined to provide accurate tissue/organ 106 oxygenation measurements both at the surface of the tissue/organ as well as deeper within the tissue. This configuration may advantageously reduce the number of tools required to obtain these measurements, potentially making obtaining the measurements less cumbersome, faster, and more accurately.

### 7.1.8. Diffuse Correlation Spectroscopy

One form of spectroscopic analysis which can be used to measure blood flow is diffuse correlation spectroscopy (DCS). DCS uses near-infrared light to non-invasively measure tissue blood flow, however DCS has limited utility as it cannot be used to provide accurate information on tissue oxygenation, or blood volume. Accordingly, in some examples DCS may be combined with near-infrared spectroscopy, to provide more detailed information on blood flow dynamics in addition to the tissue oxygenation information.

As DCS spectroscopy and near-infrared spectroscopy both use the near infrared spectrum, it may be possible to provide a measurement device 104 with a single near infrared stimulus generator 202, while providing both DCS and near-infrared spectroscopy. It should be appreciated however that a device which relies exclusively on analysis of the infra-red spectrum would have limited measurement depth characteristics as described herein.

Accordingly, in another example of the technology, a measurement device 104 may be provided which combines DCS spectroscopy and near-infrared spectroscopy with Raman spectroscopy, in order to provide blood analysis capabilities that include tissue oxygenation and vascularity, as a plurality of depths in a tissue or organ.

### 7.1.9. Display

In certain forms, the display 210 may include any means for visually communicating information to the user 102, including but not limited to, a television, monitor, segmented display, coloured lights, or LEDs. In one example of the technology, the measurement device 104 may be configured to provide a percentage readout of the tissue oxygen saturation level from any one or more of the spectral analyses performed by the measurement devices and systems described herein. For example, the display may simultaneously display tissue oxygen saturation measurements obtained from Raman and near infrared spectral analysis. In other examples the display may be selectable between two or more tissue oxygen saturation measurements, or periodically alternate between two or more tissue oxygen saturation measurements.

In another form of the technology, the display 210 may provide a simplified indication of the tissue oxygenation levels. For example, the display 210 may include a simple indication of whether the tissue oxygen levels are above or below a predefined threshold such as 60%. For example, tissue oxygen levels above 60% may be communicated with a light, such as a green status light, or text on the display such as “Good” or “OK” while oxygen levels below 60% may be communicated with a light such as a red status light, or text on the display such as “Low” or “Warning” or “Bad”.

In some examples of the technology, the display may be accompanied by one or more audible tones or tactile feedback such that the user can get feedback on the oxygen levels without needing to constantly monitor the indication on the display.

### 7.1.10. Power Source

In certain forms, the power source may include any suitable power source known to those skilled in the art, including but not limited to batteries (such as lithium batteries), alternating current (AC) sources, or direct current (DC) sources.

In some examples of the technology, such as those shown in FIG. 3. The measurement device may be passive, i.e., contain no active electronics or a power source, and instead the system 300 may be powered externally, and the light communicated to/from the measurement device using the communications interface. In this example, the system may include an AC, DC, or battery source externally to the measurement device 104.

### 7.1.11. Communications Interface

In some examples of the technology, the measurement device 104 is a standalone portable device, such as shown in FIG. 4. In this example, the measurement device may be provided with a port configured to receive a communications interface 304, such as a USB port which should be familiar to those skilled in the art. In this example the communications interface may be used for charging the internal power source, and/or communicating the measurements from the measurement device 104 to an external device such as a computer, or display.

In an alternative example the measurement device of FIG. 4, may include a wireless communications interface 304. For example, the oxygen measurements may be communicated wirelessly using Bluetooth, Wi-Fi, or any other suitable wireless technology known to those skilled in the art.

In other examples of the technology, such as those shown in FIG. 3 the communications interface 304, may be configured to communicate stimulus such as light to and from the measurement device. Accordingly, the communications interface 304 may comprise one or more light guides 504, such as optical fibres.

In other examples of the technology, any one or more of the components of the technology may be included in the measurement device 104. Accordingly, it should be appreciated that the communications interface may be configured to transfer one or more of:
Light, for example using light guides;
Power, for example using electrical conductors; and
Data, for example using any suitable communications protocol operating over one or more conductors, such as a serial, USB, I²C, SPI, CANBUS or any other suitable communications protocol known to those skilled in the art.

### 7.2. Examples of the Technology

7.2.1. Raman and Near Infrared spectroscopy Measurement

The present technology provides measurement devices and systems which combine two or more spectral analysis technologies for measuring oxygen saturation within an organ or tissue.

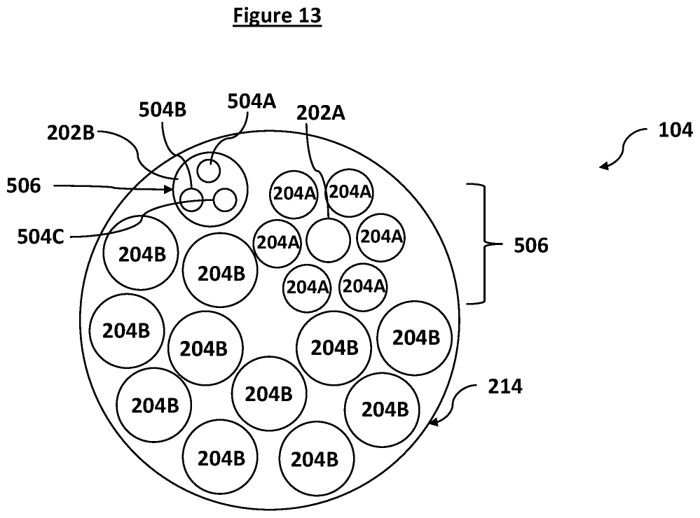

FIG. 13 shows one example cross-section of the end of a measurement device which comprises Raman spectroscopy and near infrared spectroscopy technologies. As shown the measurement device 104 comprises a plurality of stimulus generators 202A, 202B, or is otherwise optically coupled to a plurality of stimulus generators, for example using the light guides 504 described herein. In this example, stimulus generator 202A is preferably configured to generate a light source suitable for the determination of blood-oxygen concentrations using Raman analysis such as a substantially 410 nm wavelength light, as described herein.

Surrounding the Raman light source 202A is a plurality of signal acquisition unit(s) 202A or a plurality of light guides 504 or optical fibres optically connected to one or more signal acquisition units. Accordingly, the Raman analysis part of the measurement device may substantially correspond to the example of FIG. 11.

Accordingly, a first optical sub-assembly 506 is provided by the arrangement of the stimulus generator 202A, and signal acquisition unit 204A, and/or the light guides 504 thereof. In this example the first optical sub-assembly is configured for use in performing Raman spectroscopy.

Also provided is a stimulus generator 202B configured to generate a light source suitable for the determination of blood-oxygen concentrations using near infrared spectral analysis such as the substantially 785 nm, substantially 810 nm and substantially 830 nm wavelength light sources described herein. In the example shown, three separate light guides 504 are provided, each light guide being configured to transfer one of the respective light sources. As noted above, different wavelength light sources, can be used to perform oxygen measurements at different depths within the organ/tissue 106. Accordingly, the use of three different wavelengths should not be seen as limiting on the technology, and a single wavelength of light may be used for near infrared spectral analysis. Additionally, the use of a plurality of light guides 504A, 504B, 504C should not be seen as limiting, and in other examples wherein the light sources are provided at discrete time intervals as shown in FIG. 6, a single light guide and/or stimulus generator 202 may be used to generate each of the plurality of wavelengths.

In the configuration shown in FIG. 13, it can be seen that the stimulus generator 202B tasked with generating light in the near infrared range is positioned substantially towards one side of the measurement device 104 housing 214. This configuration allows for a range of signal acquisition units/light guides 204B to be positioned at varying radial distances from the stimulus generator 202B, to thereby allow for measurements to be taken at a plurality of organ/tissue depths.

Accordingly, a second optical sub-assembly 506 is provided by the arrangement of the stimulus generator 202B, and signal acquisition unit 204B, and/or the light guides 504 thereof. In this example the second optical sub-assembly 506 is configured for use in performing near infrared spectroscopy.

For completeness we note that the example of FIG. 13 comprises a first stimulus generator/light guide optically connected to a stimulus generator 202A configured to generate a light source suitable for blood oxygen measurement using Raman spectroscopy, and a plurality (six in this example, but this should not be seen as limiting) signal acquisition units/light guides 204A. The example also comprises a second stimulus generator/light guide optically connected to a stimulus generator 202B configured to generate a light source suitable for blood oxygen measurement using near infrared spectroscopy, and a plurality (eleven in this example) signal acquisition units/light guides 204B.

Accordingly, by using a plurality of signal acquisition units/light guides 204B in a measurement device, it may be possible to increase the total light/signal strength received by the measurement device, which may be able to improve the accuracy of the tissue oxygen measurements. In some examples two or more signal acquisition units/light guides 204B may be positioned at substantially the same distance (i.e., radial distance) from the stimulus generator to increase the amount of light received by the signal acquisition units/light guides 204B at that distance.

As described herein, the distance of the signal acquisition units/light guides 204B relative to the stimulus generator is believed to directly relate to the depth at which the measurement is being performed in the tissue. Accordingly, as the distance and depth increases, it may be advantageous to include additional signal acquisition units/light guides 204B to receive more of the signal which is attenuated by the surrounding tissues. Accordingly, in some examples of the technology, it may be advantageous to include additional signal acquisition units/light guides 204B as the radial distance from the stimulus generator increases. For example, a first set of signal acquisition units/light guides 204B may be provided at a first radial distance from the stimulus generator, and a second set of signal acquisition units/light guides 204B may be provided at a second radial distance from the stimulus generator, wherein the second radial distance is greater than the first radial distance and the second set of signal acquisition units/light guides includes more signal acquisition units/light guides than the first set.

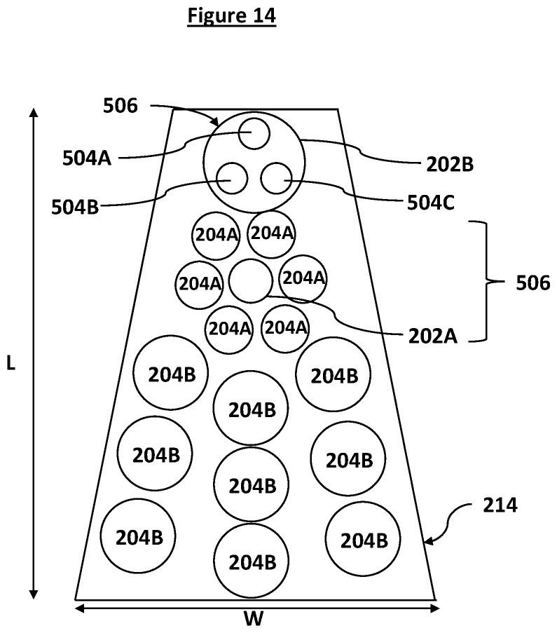

While the example shown in FIG. 13 is provided with a substantially circular housing, this should not be seen as limiting on the technology as described herein. For example, FIG. 14, shows an alternative configuration of a measuring device 104 in accordance with the present technology which comprises using a polygonal housing 214 which has a length which is greater than its width. For example, the substantially trapezoidal cross section shown in FIG. 14, or any other suitable polygonal shape such as triangular, quadrilateral, pentagonal, hexagonal, octagonal, etc. In a similar manner to the example of FIG. 13, the stimulus generator/light guides 202B tasked with generating near infrared light, is positioned on a first side/end of the housing 214, so as to allow for maximum separation from the corresponding acquisition units/light guides 204B.

In the examples of FIGS. 13 and 14, the second stimulus generator 202B used for near infrared spectroscopy is positioned on a first side of the measurement device 104, and in some examples the first stimulus generator 204A for the Raman spectroscopy, is positioned adjacent to the second stimulus generator 202B so as to allow for greater separation between the second stimulus generator 202B and the corresponding signal acquisition units, or light guides therefor. In other words, the first optical sub-assembly 506 for performing Raman spectroscopy may be positioned within the second sub-assembly 506 for performing near infrared spectroscopy, such as between the stimulus generator 202B, and the signal acquisition units 204B or the light guides 504 thereof.

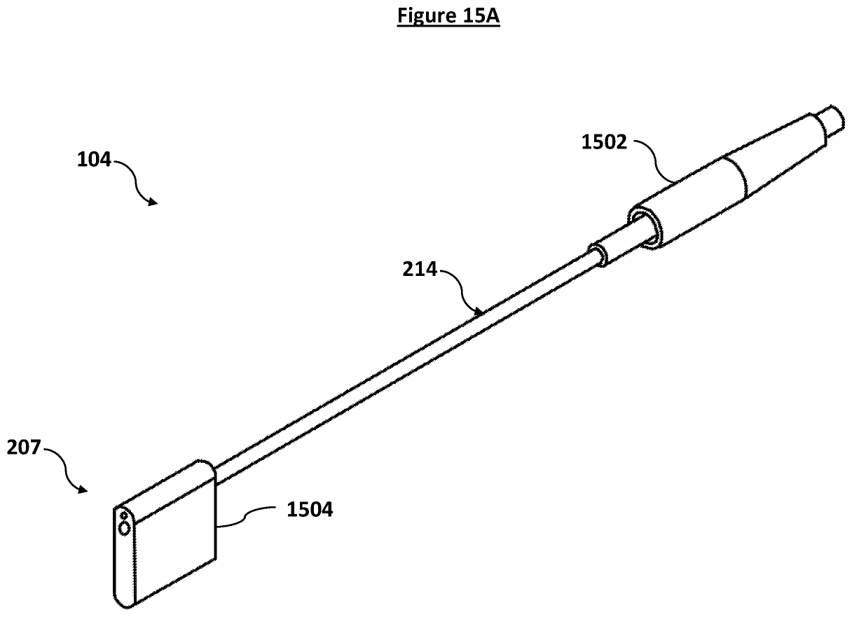

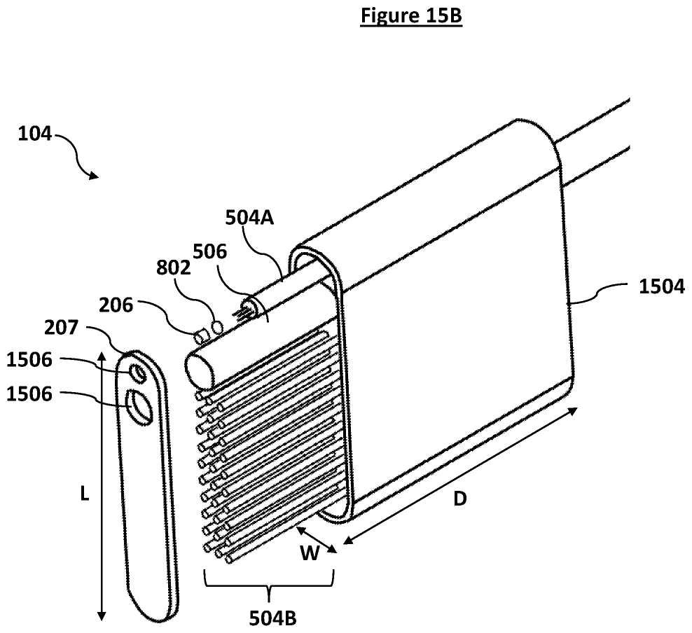

FIGS. 15A and 15B show a further example of a measurement device 104 in accordance with the present technology.

In these examples the measurement device 104 comprises a substantially elongate cylindrical housing 214, and a handle 1502 which in use is gripped by the user. In the illustrated example the handle has a substantially circular cross section, or is otherwise cylindrical, however in other examples this may be shaped to conform with a user's hand or have any other suitable shape, such as hexagonal.

At the first (distal) end 207 of the measurement device the housing 214 transitions or is otherwise connected to a measurement tip 1504, which is shown in an exploded view in FIG. 15B. The illustrated measurement tip has a cross-sectional profile of a rounded corner rectangle, or lozenge, although it should be appreciated that the use of rounded corners is optional, and the measurement tip 1504, may instead be substantially rectangular.

It has been described earlier that, in some forms, the area of the measurement tip (which is measurement tip 1504 in the example of FIGS. 15A and 15B) may be no bigger than approximately 80 mm². It will be appreciated that a suitably small area of the tip may be achieved by the dimensions of the tip. For example, the profile of the measurement tip 1504 is configured to have a length ‘L’ which may allow for a substantial separation between the light guide 504A carrying the near infrared stimulus, and the light guides 504B configured to detect the received near infrared light from the tissues and/or organs of the patient, aiming to increase that separation while balancing this with the desired to reduce the overall size of the measurement tip 1504 for ease of use and compactness. For example, the measurement tip 1504 may have a length ‘L’ of between approximately 20 mm, and approximately 35 mm, such as approximately 25 mm, so as to allow for the stimulus light guide 504A to be separate from the light guides receiving the light 504B of up to approximately 20 mm to allow tissue/organ depth measurements of up to approximately 30 mm as described herein.

The measurement tip 1504 may further have a width ‘W’ of between approximately 1 mm and approximately 15 mm, such as approximately 10 mm. Use of an approximately 10 mm wide measurement tip, may advantageously allow for multiple columns of light guides 504B to be included allowing for a cone or window of measurement coverage on the tissue/organ as described herein. In one exemplary form, the measurement tip 1504 may have a length of approximately 20 mm and a width of approximately 5 mm, providing an area of approximately 30 mm². In other forms, any one or more of these quantities may be less.

The measurement tip 1504 may further have a depth ‘D’ of between approximately 30 mm and approximately 150 mm, such as approximately 100 mm. Use of an approximately 100 mm deep housing may advantageously allow for routing of the light guides 504 within the housing, without exceeding the critical bending radii (i.e., having a radius tighter or less than the critical bending radii) for the light guides/optical fibres 504, while also allowing practical hand control for surgeons.

As in the previous examples, the near infrared stimulus is provided at a first side of the housing, so as to enable a high amount of separation between the outgoing and incoming near infrared light for the physical size of the measurement tip 1504. The Raman optical subassembly 506 (shown as a single member for simplicity) is positioned between the outgoing near infrared light guides 504A and the incoming near infrared light guides 504B. For completeness the Raman optical subassembly 506 can have any suitable configuration including a cross sectional configuration such as the example shown in FIG. 11, and/or an integrated transmission material 206, such as the example illustrated in FIG. 8.

In the illustrated example, ten rows of three light guides 504B are arranged such that each row is provided with a greater distance, e.g., greater radial distance, from the light guide 504A carrying the near infrared stimulus, thereby allowing for a range of depth measurements within the tissue/organ. The use of a plurality of light guides in each row may allow for additional light to be captured at each distance from the light guide 504A carrying the near infrared stimulus. This can help to account for a reduction in light intensity coming back from the tissue and may improve the sensitivity of the probe. In the example shown, a removable first end 207 (which may be a transmission medium 206) is provided which substantially conforms to the shape of the measurement tip 1504, housing so as to provide a seal which protects the light guides 504, and provides a seal (such as a hermetic seal) to the measurement tip to protect the light guides from the tissue, and makes the measurement device 104, safe for/resilient against damage during sterilisation. In this example, the transmission medium is provided with one or more openings 1506 corresponding to any one or more of the:
Near infrared light guides 504A associated with outgoing light from the stimulus generator(s);
Raman light guides (inside optical sub-assembly 506) associated with the outgoing light from the stimulus generator(s); and/or
Raman light guides (inside optical sub-assembly 506) associated with the incoming light from the tissue/organs.

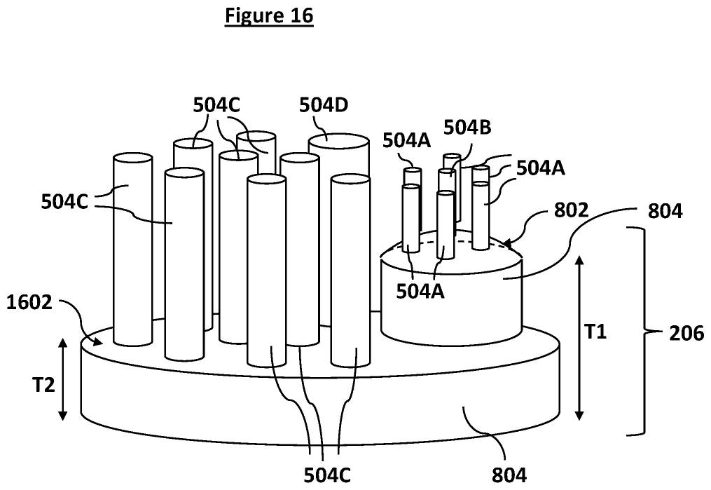

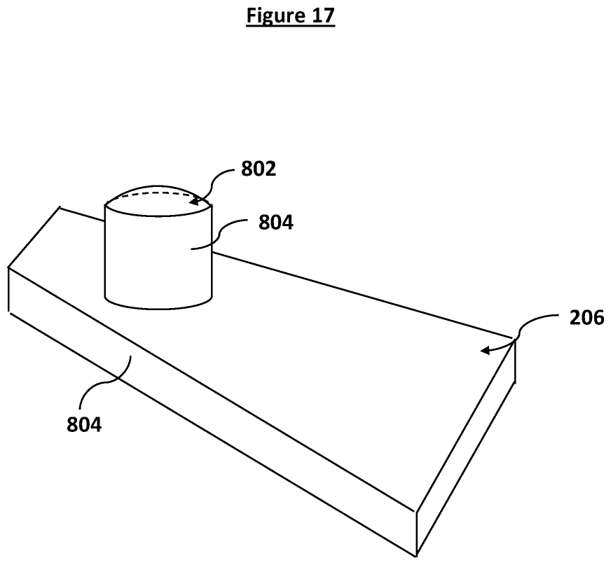

In other words, any one or more of the light guides may be provided with or without a transmission medium. Additionally, the transmission medium may be configured to be provided as multiple components, or with varying dimensions, as shown in FIG. 15B. For example, the transmission medium provided for Raman spectroscopy may be different to the transmission medium provided for near infrared spectroscopy, as will be further explained in relation to FIGS. 16 and 17.

### 7.2.1.1. Complex Transmission Media

In the examples described herein, transmission media, lenses and transmission windows have been described in relation to Raman and near infrared spectral analysis. Accordingly, in examples of the technology comprising both Raman and near infrared spectral analysis, it may be advantageous to provide transmission media which is designed to account for both the Raman surface analysis and the near infrared depth analysis.

One example of a suitable transmission medium 206 for a combined measurement device 104 is shown in FIG. 16. In this example, the transmission medium 206 is designed to accommodate a substantially cylindrical housing 214, which largely corresponds to the example of FIG. 13. As shown, the transmission medium 206 comprises a lens 802, which as described in relation to previous examples of the technology, may be used to direct the received light from the organ/tissue 106 into one or more signal acquisition units 204, via the light guides 504A. Also shown is a light guide 504B which is configured to provide the stimulus for Raman analysis. Light guide 504B is preferably positioned centrally above the lens 802 such that the lens provides minimal deflection of the light, as described in relation to the previous embodiments.

The lens is provided over a raised portion of the transmission window 804, so as to allow a larger spot ‘S’ to be formed at the target area as described in relation to previous examples. The raised portion may be integral to, attached to, or otherwise separate from the rest of the transmission window.

Accordingly, in the example shown in FIG. 16, the transmission window which corresponds to the Raman analysis function is provided with a lens 802 and transmission window 804 which has a length ‘T1’, which is greater than the length of the transmission window provided to the light guides 504C, 504D which are related to performing near infrared analysis. For example, the transmission window length T1 may be between approximately 3.5 mm and approximately 1.5 mm, such as approximately 3 mm. The transmission window length T2, may be between approximately 0.25 mm and approximately 1 mm, such as approximately 0.5 mm.

In alternative examples of the technology length T1 may be substantially equal to length T2, for example this configuration may advantageously allow for a more cost-effective lens construction.

It should also be noted the transmission medium 206 associated with the near infra-red analysis is also provided with a substantially planer top surface 1602, such that there is minimal deflection of the light associated with near infrared analysis. In other words, the lens 802 is only provided to the components of the technology associated with performing Raman analysis.

Light guide 504D may contain a plurality of wavelength sources, or light guides 504 comprising a plurality of wavelengths as described in relation to FIGS. 13 and 14. While light guides 504C may be configured to transmit the detected light back to one or more signal acquisition units tasked with performing near infrared analysis.

It should be appreciated that the transmission medium 206 described herein is designed to be attached to or otherwise inserted into the first (distal) end 207 of a measurement device as described herein, such that the end of the measurement device is substantially planar, and substantially perpendicular to the longitudinal axis of the measurement device.

Similarly FIG. 17, shows one example of a transmission medium 206, which substantially corresponds to the example shown in FIG. 14. In this example the light guides 504, have been omitted by way of clarity, but it should be appreciated that light guides 504, and/or stimulus generators and signal acquisition units may be optically coupled in the approximate locations shown in FIG. 14, or any other suitable configuration known to those skilled in the art.

### 7.2.2. Other Examples

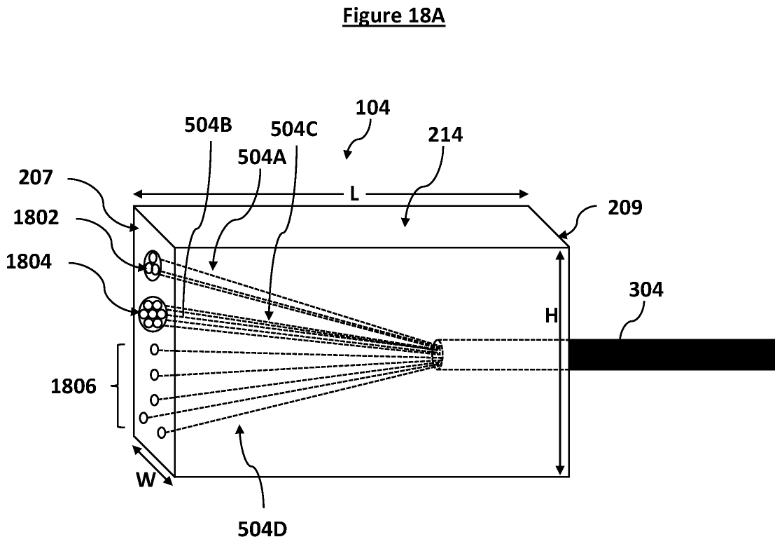

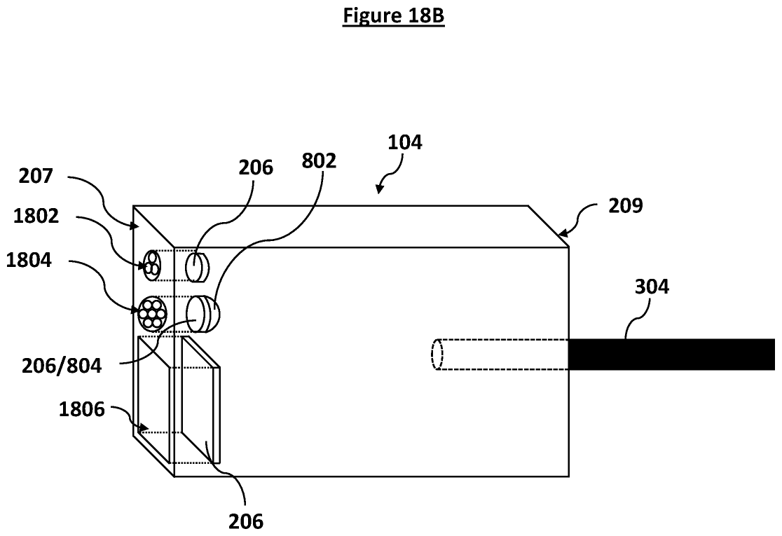

FIG. 18A shows another example of the technology in which a measurement device 104 is provided for measuring oxygen saturation levels in an organ and/or tissue. In this example the measurement device comprises a housing 214 having a first end 207 and a second end 209, the first end 207 being distal to the second end 209.

In this example the first end of the housing includes a first opening 1802 or aperture which as shown in FIG. 18B, includes at least one transmission media 206 which may be referred to as a first transmission media 206, although it should be appreciated that more than one transmission media may be used.

Optically connected to the opening first opening 1802 is a first set of light guides 504A, which are configured to receive a first source of light for example from a stimulus generator 202 which may be internal to the housing, or external to the housing as described in relation to FIGS. 2, 3A and 3B. The first source of light is directed through the transmission media 206, and out of the first opening 1082, such that it is projected onto a tissue or organ in use.

The first set of light guides shown in FIG. 18A is shown schematically as a bundle of three dashed lines which extend from the communications interface 304 to the first opening 1802. These lines have been omitted from FIG. 18B for clarity.

The measurement device 104 also includes a second opening 1804 at the first end 207, the second opening including one or more transmission media 206, which may be referred to as a second transmission media, and in some examples may be the same transmission media as the first transmission media. For example, as illustrated in FIG. 18B, the transmission media 206 includes a transmission window 804 and lens 802 as described herein (such as a plano-convex lens configured to focus the incident light into parallel light rays to be received by the light guides).

Optically connected to the second opening is a second set of light guides 504B, which are configured to receive a second source of light (for example from a stimulus generator 202 which may be internal to the housing, or external to the housing as described in relation to FIGS. 2, 3A and 3B) and direct the second source of light through the one or more transmission media 206 of the second opening 1804 and out of the second opening that it is projected onto a tissue or organ in use.

The second set of light guides 504B shown schematically in FIG. 18A refers to the light guides connected to the centre of the second opening 1804, which transfers light from the communications interface 304 to the second opening 1804. This arrangement substantially mirrors that shown in FIG. 11. In the illustrated example this is a single light guide, however more than one light guide may be used.

Also illustrated schematically is a third set of light guides 504C configured to receive a third source of light through the second opening (such as reflected/refracted light from the tissue), and to direct the third source of light into a signal acquisition unit 204 and/or processor 208 configured to perform a Raman spectral analysis of the third source of light. The third set of light guides 504C may be positioned to receive the third source of light at an end of the light guides 504C at the distal end of the measurement device 104.

The third set of light guides 504C shown schematically in FIG. 18A refers to the light guides connected to the second opening which surround the second set of light guides 504B. These light guides transfer incident light reflected or refracted from the tissue/organ, into the measurement device and transfer these to the signal acquisition unit 204 and/or processor 208 configured to perform a Raman spectral analysis.

The measurement device 104 also includes a third set of openings 1806, which in FIG. 18A are shown as a plurality (five) substantially circular openings, while in FIG. 18B this is shown as a single substantially rectangular opening. The third set of openings 1806 includes one or more transmission media 206, which may be referred to as a third transmission media, and in some examples may be the same transmission media as either of the first and second transmission media, or a transmission media which is common to all light guides. In other words the transmission media 206 through which the first, second, third or fourth sources of light are directed may be the same as or different to the transmission media through which any of the other of the first, second, third or fourth sources of light are directed.

Optically coupled to the third set of openings is a fourth set of light guides (indicated schematically as dashed lines 504D) configured to receive a fourth source of light through the third set of one or more openings 1806, and to direct the fourth source of light, into a signal acquisition unit 204 and/or processor 208 (internal or external to the measurement device 104) configured to perform visible and/or near-infrared spectral analysis of the fourth source of light. The fourth set of light guides 504D may be positioned to receive the fourth source of light at an end of the light guides 504C at the distal end of the measurement device 104.

It should be appreciated that the first end 207 of the measurement device 104 may be substantially opaque to light, while the openings 1802, 1804, 1806 and associated transmission media 206 may provide a pathway for the transmission of light into and out of the measurement device 104. In some examples these transmission media 204 may be configured to sit substantially flush with the front surface of the first end 207, such that there are no localised pressure points created when the first end 207 contacts the tissue or organ in use.

It should also be understood that the fourth source of light used in the visible and/or near infrared spectral analysis substantially arises through an interaction of the organ and/or tissue with the first source of light, for example via reflection, refraction or scattering of the light. Similarly, the third source of light used in the Raman spectral analysis substantially arises through an interaction of the organ and/or tissue with the second source of light.

In the illustrated example of FIGS. 18A and 18B the measurement device may have a length (‘L’) a width (‘W’) and a height (‘H’) as described herein. For example, the length may be between approximately 10 cm and approximately 50 cm such as approximately 30 cm. The width may be between approximately 0.5 cm and approximately 2 cm such as approximately 1 cm, and the height may be between approximately 3 cm and 1 cm, such as approximately 1.6 cm.

### 7.3. Other Remarks

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The technology may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the technology and without diminishing its attendant advantages. It is therefore intended that such changes and modifications be included within the present technology.

1. A measurement device for use in measuring oxygen saturation levels in an organ and/or tissue, the measurement device comprising:
a housing having a first end and a second end, the first end being distal to the second end, and the housing comprising:
a first set of light guides configured to receive a first source of light, and direct the first source of light through one or more transmission media and out of the first end,
a second set of light guides configured to receive a second source of light, and direct the second source of light through one or more transmission media and out of the first end,
a third set of light guides configured to receive a third source of light, and to direct the third source of light into a signal acquisition unit and/or processor configured to perform a Raman spectral analysis of the third source of light,
a fourth set of light guides configured to receive a fourth source of light, and to direct the fourth source of light into a signal acquisition unit and/or processor configured to perform visible and/or near-infrared spectral analysis of the fourth source of light.

2. The measurement device as claimed in claim 1, wherein the first source of light comprises one or more wavelengths between 700 nm and 900 nm.

3. The measurement device as claimed in claim 1, wherein the first source of light comprises one or more wavelengths, each wavelength independently selected from the group consisting of: between approximately 700 nm and approximately 800 nm, between approximately 795 nm and approximately 825 nm, and between approximately 815 nm and approximately 900 nm.

4. The measurement device as claimed in claim 1, wherein the first source of light has one or more wavelengths of light selected from the group consisting of: approximately 785 nm, approximately 810 nm and approximately 830 nm.

5. The measurement device as claimed in claim 1, wherein the first set of light guides comprise a plurality of light guides, and wherein a first light guide of the plurality of light guides is configured to receive light having a wavelength of between approximately 700 nm and approximately 800 nm, a second light guide of the plurality of light guides is configured to receive light having a wavelength of between approximately 795 nm and approximately 825 nm, and a third light guide of the plurality of light guides is configured to receive light having a wavelength of between approximately 815 nm and approximately 900 nm.

6. (canceled)

7. The measurement device as claimed in claim 1, wherein the second source of light has a wavelength of between approximately 380 nm and approximately 425 nm.

8. (canceled)

9. (canceled)

10. The measurement device as claimed in claim 1, wherein one or more of the transmission media comprises a lens, a window or a filter.

11. The measurement device as claimed in claim 10, wherein the lens is a plano-convex lens.

12. (canceled)

13. (canceled)

14. (canceled)

15. (canceled)

16. (canceled)

17. The measurement device as claimed in claim 1, wherein the first, second, third and/or fourth light guides comprise one or more optical fibres and wherein the third set of light guides comprises three or more optical fibres.

18. (canceled)

19. The measurement device as claimed in claim 1, wherein the first, second, third and/or fourth light guides comprise one or more optical fibres and wherein the fourth set of light guides comprises three or more optical fibres.

20. (canceled)

21. The measurement device as claimed in claim 1, wherein the first source of light and the second source of light are provided by one or more stimulus generators, wherein the stimulus generators each have a power of less than approximately 50 mW.

22. (canceled)

23. (canceled)

24. (canceled)

25. (canceled)

26. The measurement device as claimed in claim 1, wherein the Raman spectral analysis and/or the visible and/or near infrared spectral analysis are used to determine the oxygen saturation levels in the tissue or organ.

27. The measurement device as claimed in claim 1, further comprising a display configured to provide an indication of the oxygen saturation levels.

28. The measurement device as claimed in claim 1, wherein the measurement device is a handheld device.

29. The measurement device as claimed in claim 1, wherein the measurement device is connected to physically and/or remotely to a data collection and/or processing unit.

30. The measurement device as claimed in claim 1, wherein the first end is configured to be positioned in contact with, or in close proximity to, the organ and/or tissue in use, wherein an area of the first end is 100 mm² or less.

31. The measurement device as claimed in claim 30, wherein the first end has:
a) a length, a measurement tip and a width, and wherein the length of the measurement tip is 20 mm or less, or
b) a length of between 20 mm and 35 mm.

32. (canceled)

33. The measurement device as claimed in claim 30, wherein the width of the first end is 5 mm or less.

34. The measurement device as claimed in claim 30, wherein the width of the first end is between 5 mm and 25 mm.

35. (canceled)

36. (canceled)

37. The measurement device as claimed in claim 1, wherein the housing comprises:
a) a third opening at the first end, wherein one or more transmission media through which the third source of light is directed is positioned in the third opening, or
b) one or more fourth openings at the first end, wherein one or more transmission media through which the fourth source of light is directed is positioned in the first opening.

38. (canceled)

39-42. (canceled)
